SEQUENCE LISTING (139 sequences)

SEQ ID NO: 1 - DNA, 37 bp, Homo sapiens
cttaaaagcttaagcttctctcgcagaaatgccaaat

SEQ ID NO: 2 - DNA, 67 bp, Homo sapiens
cttaatctagaaaaaaaatggtcttcagtgaagcgaagacgcagggttcgaacctgcgcggggagac

SEQ ID NO: 3 - DNA, 30 bp, Homo sapiens
ccctgctcgctgcggaagcgggtgctctta

SEQ ID NO: 4 - DNA, 30 bp, Homo sapiens
aaaataagagcacccgcttccgcagcgagc

SEQ ID NO: 5 - DNA, 33 bp, Homo sapiens
ccctgctcgctgcgttcagcccgtcctctaggc

SEQ ID NO: 6 - DNA, 33 bp, Homo sapiens
aaaagcctagaggacgggctgaacgcagcgagc

SEQ ID NO: 7 - DNA, 33 bp, Homo sapiens
ccctgctcgctgcgctcctcgagcgtcagacgc

SEQ ID NO: 8 - DNA, 33 bp, Homo sapiens
aaaagcgtctgacgctcgaggagcgcagcgagc

SEQ ID NO: 9 - DNA, 32 bp, Homo sapiens
ccctgctcgctgcggagaagcgggtgctctta

SEQ ID NO: 10 - DNA, 32 bp, Homo sapiens
aaaataagagcacccgcttctccgcagcgagc

SEQ ID NO: 11 - DNA, 34 bp, Homo sapiens
ccctgctcgctgcggagagaagcgggtgctctta

SEQ ID NO: 12 - DNA, 34 bp, Homo sapiens
aaaataagagcacccgcttctctccgcagcgagc

SEQ ID NO: 13 - DNA, 28 bp, Homo sapiens
ccctgctcgctgcgagcgggtgctctta

SEQ ID NO: 14 - DNA, 28 bp, Homo sapiens
aaaataagagcacccgctcgcagcgagc

SEQ ID NO: 15 - DNA, 19 bp, Homo sapiens
gcacatggttagatcaagc

SEQ ID NO: 16 - DNA, 22 bp, Homo sapiens
gaaaacccacaatccctggctg

SEQ ID NO: 17 - DNA, 23 bp, Homo sapiens
gaaaacccacaatccctggctta

SEQ ID NO: 18 - DNA, 24 bp, Homo sapiens
gtcaattagttgtaaacaccactg

SEQ ID NO: 19 - DNA, 22 bp, Homo sapiens
gttctaggataggcccaggggc

SEQ ID NO: 20 - DNA, 20 bp, Homo sapiens
gccaactgagctaaccggcc

SEQ ID NO: 21 - DNA, 24 bp, Homo sapiens
gaaccccaccaacatagggcttcg

SEQ ID NO: 22 - DNA, 22 bp, Homo sapiens
gggcaggcgagaattctaccac

SEQ ID NO: 23 - DNA, 22 bp, Homo sapiens
ggataaccactacactatggaa

SEQ ID NO: 24 - DNA, 20 bp, Homo sapiens
gtggcgcccgaacagggaca

SEQ ID NO: 25 - DNA, 21 bp, Homo sapiens
ggcaccccagatgggacacga

SEQ ID NO: 26 - DNA, 19 bp, Homo sapiens
gaaacgaggtaactccgga

SEQ ID NO: 27 - DNA, 23 bp, Homo sapiens
gtgcccgagtgtggtggagaatg

SEQ ID NO: 28 - DNA, 21 bp, Homo sapiens
gagtagtggtgcgttggccgg

SEQ ID NO: 29 - DNA, 20 bp, Homo sapiens
gtggcgaccacgaagggacg

SEQ ID NO: 30 - DNA, 23 bp, Homo sapiens
gaattctaccactgaaccacaat

SEQ ID NO: 31 - DNA, 21 bp, Homo sapiens
ggcgaccacgaagggacacga

SEQ ID NO: 32 - DNA, 20 bp, Homo sapiens
gttgtaaacaccactgcact

SEQ ID NO: 33 - DNA, 22 bp, Homo sapiens
gggcttcaaaaaatttgcttga

SEQ ID NO: 34 - DNA, 21 bp, Homo sapiens
ggagggggcacccggatttga

SEQ ID NO: 35 - DNA, 21 bp, Homo sapiens
ggcgaccacgaagggacccga

SEQ ID NO: 36 - DNA, 21 bp, Homo sapiens
gactacctgcactataagcac

SEQ ID NO: 37 - DNA, 21 bp, Homo sapiens
ggtgcgttggccgggaaacga

SEQ ID NO: 38 - DNA, 21 bp, Homo sapiens
gaaacagcaagctagtcaagc

SEQ ID NO: 39 - DNA, 23 bp, Homo sapiens
gcttagaccgctcggccatcctt

SEQ ID NO: 40 - DNA, 23 bp, Homo sapiens
gaccgctcggccacgctaccctc

SEQ ID NO: 41 - DNA, 19 bp, Homo sapiens
gtggcgagccagccaggag

SEQ ID NO: 42 - DNA, 23 bp, Homo sapiens
gccttagaccgctcggccatcct

SEQ ID NO: 43 - DNA, 23 bp, Homo sapiens
gtccttggtgcccgaggtgtcta

SEQ ID NO: 44 - DNA, 22 bp, Homo sapiens
gtgatatccactacactacgga

SEQ ID NO: 45 - DNA, 21 bp, Homo sapiens
gcaccactataccaccaacgc

SEQ ID NO: 46 - DNA, 23 bp, Homo sapiens
gctcgccagggcaaggcttacaa

SEQ ID NO: 47 - DNA, 20 bp, Homo sapiens
ggtgcatgggccgggaaacg

SEQ ID NO: 48 - DNA, 20 bp, Homo sapiens
gaccactgaaccaccaatgc

SEQ ID NO: 49 - DNA, 22 bp, Homo sapiens
ggttcctgaccgggaatcgaac

SEQ ID NO: 50 - DNA, 21 bp, Homo sapiens
ggtgccgaaacccgggaacga

SEQ ID NO: 51 - DNA, 22 bp, Homo sapiens
gaaccccaccaacatagggctt

SEQ ID NO: 52 - DNA, 22 bp, Homo sapiens
gtccttggtgcccgagtgacct

SEQ ID NO: 53 - DNA, 22 bp, Homo sapiens
gacaccgtccttggtgccgcgt

SEQ ID NO: 54 - DNA, 22 bp, Homo sapiens
ggacaccgtccttggtgcccag

SEQ ID NO: 55 - DNA, 22 bp, Homo sapiens
gcccgaggtggtatggccgtag

SEQ ID NO: 56 - DNA, 22 bp, Homo sapiens
gtctaccactgaaccacccatg

SEQ ID NO: 57 - DNA, 20 bp, Homo sapiens
gaccactgaaccacccatgc

SEQ ID NO: 58 - DNA, 22 bp, Homo sapiens
gagaaccgtccttggtgcccga

SEQ ID NO: 59 - DNA, 21 bp, Homo sapiens
gaaaataagagcacccgcttc

SEQ ID NO: 60 - DNA, 18 bp, Homo sapiens
gcgaggtaactccggagc

SEQ ID NO: 61 - DNA, 19 bp, Homo sapiens
gaggcacctgccaggtgac

SEQ ID NO: 62 - DNA, 20 bp, Homo sapiens
gctgagcacaggacttcctt

SEQ ID NO: 63 - DNA, 20 bp, Homo sapiens
gagcttggacgctcggttga

SEQ ID NO: 64 - DNA, 23 bp, Homo sapiens
gtcgccctctcaacccagctttt

SEQ ID NO: 65 - DNA, 23 bp, Homo sapiens
gatcgggaggggactgagcctga

SEQ ID NO: 66 - DNA, 20 bp, Homo sapiens
gtcctggaaaccaggagtgc

SEQ ID NO: 67 - DNA, 22 bp, Homo sapiens
aactatacaacctactacctca

SEQ ID NO: 68 - DNA, 21 bp, Homo sapiens
acaaaccattatgtgctgcta

SEQ ID NO: 69 - DNA, 22 bp, Homo sapiens
gctatctgcactagatgcacct

SEQ ID NO: 70 - DNA, 21 bp, Homo sapiens
gtcaacatcagtctgataagc

SEQ ID NO: 71 - DNA, 22 bp, Homo sapiens
gtcatagccctgtacaatgctg

SEQ ID NO: 72 - DNA, 23 bp, Homo sapiens
gctacctgcactgtaagcacttt

SEQ ID NO: 73 - DNA, 23 bp, Homo sapiens
gcagctgcttttgggattccgtt

SEQ ID NO: 74 - DNA, 23 bp, Homo sapiens
gccatgctaatcttctctgtatc

SEQ ID NO: 75 - DNA, 24 bp, Homo sapiens
agaactggacaaagttttcatcac

SEQ ID NO: 76 - DNA, 19 bp, Homo sapiens
gcctagaggacgggctgaa

SEQ ID NO: 77 - DNA, 19 bp, Homo sapiens
gcgtctgacgctcgaggag

SEQ ID NO: 78 - DNA, 23 bp, Homo sapiens
ggcggcagtcctcagtactctta

SEQ ID NO: 79 - DNA, 17 bp, Homo sapiens
ctgtaggcaccatcaat

SEQ ID NO: 80 - DNA, 30 bp, Homo sapiens
cttaaaagcttactaaagtgtctccgcctg

SEQ ID NO: 81 - DNA, 50 bp, Homo sapiens
aaataagagcacccgcttccgcagcgagcagggttcgaacctgcgcgggg

SEQ ID NO: 82 - DNA, 59 bp, Homo sapiens
tcgacgagtagtggtgcgttggccgggaaaaaaacgagtagtggtgcgttggccgggaa

SEQ ID NO: 83 - DNA, 59 bp, Homo sapiens
ctagttcccggccaacgcaccactactcgtttttttcccggccaacgcaccactactcg

SEQ ID NO: 84 - DNA, 48 bp, Homo sapiens
tcgaaaaataagagcacccgcttcaaaaaaaataagagcacccgcttc

SEQ ID NO: 85 - DNA, 48 bp, Homo sapiens
ctaggaagcgggtgctcttattttttttgaagcgggtgctcttatttt

SEQ ID NO: 86 - DNA, 24 bp, Homo sapiens
tcgaaaaataagagcacccgcttc

SEQ ID NO: 87 - DNA, 24 bp, Homo sapiens
ctaggaagcgggtgctcttatttt

SEQ ID NO: 88 - DNA, 64 bp, Homo sapiens
tcgaaaaaaaaataagagcacccgcttctctcaaaaaaaaaaaataagagcacccgcttctctc

SEQ ID NO: 89 - DNA, 64 bp, Homo sapiens
ctaggagagaagcgggtgctcttattttttttttttgagagaagcgggtgctcttatttttttt

SEQ ID NO: 90 - DNA, 81 bp, Homo sapiens
tcgaaactatacaacctactacctcaaaaaaaactatacaacctactacctcaaaaaaaaactatacaacctactacctca

SEQ ID NO: 91 - DNA, 81 bp, Homo sapiens
ctagtgaggtagtaggttgtatagtttttttttgaggtagtaggttgtatagttttttttgaggtagtaggttgtatagtt

SEQ ID NO: 92 - DNA, 84 bp, Homo sapiens
tcgaatctattgaaggaactacctcaaaaaaaaatctattgaaggaactacctcaaaaaaaaatctattgaaggaactacctca

SEQ ID NO: 93 - DNA, 84 bp, Homo sapiens
ctagtgaggtagttccttcaatagatttttttttgaggtagttccttcaatagatttttttttgaggtagttccttcaatagat

SEQ ID NO: 94 - DNA, 85 bp, Homo sapiens
tcgactacctgcactataagcactttaaaaaaactacctgcactataagcactttaaaaaaactacctgcactataagcacttta

SEQ ID NO: 95 - DNA, 85 bp, Homo sapiens
ctagtaaagtgcttatagtgcaggtagtttttttaaagtgcttatagtgcaggtagtttttttaaagtgcttatagtgcaggtag

SEQ ID NO: 96 - DNA, 85 bp, Homo sapiens
tcgacttgctgcagtataagcactttaaaaaaacttgctgcagtataagcactttaaaaaaacttgctgcagtataagcacttta

SEQ ID NO: 97 - DNA, 85 bp, Homo sapiens
ctagtaaagtgcttatactgcagcaagtttttttaaagtgcttatactgcagcaagtttttttaaagtgcttatactgcagcaag

SEQ ID NO: 98 - DNA, 88 bp, Homo sapiens
tcgatagttttcacaatgatctcggtagttttcacaatgatctcggtagttttcacaatgatctcggtagttttcacaatgatctcgg

SEQ ID NO: 99 - DNA, 88 bp, Homo sapiens
ctagccgagatcattgtgaaaactaccgagatcattgtgaaaactaccgagatcattgtgaaaactaccgagatcattgtgaaaacta

SEQ ID NO: 100 - DNA, 22 bp, Homo sapiens
tagtggmugmcgmutmggccgg

SEQ ID NO: 101 - DNA, 25 bp, Homo sapiens
aaaatmaamgamgcmacmccgcttc

SEQ ID NO: 102 - RNA, 40 nt, Homo sapiens
mamamamamumamamgmamgmcmamcmcmcmgmcmumumc

SEQ ID NO: 103 - RNA, 20 nt, Homo sapiens
aaaauaagagcacccgcuuc

SEQ ID NO: 104 - DNA, 32 bp, Homo sapiens
aatggcctcgagcctcctcaattcacaacctg

SEQ ID NO: 105 - RNA, 48 nt, Homo sapiens
mamgmgmcmgmgmcmamgmumcmcmumcmamgmumamcmumcmumuma

SEQ ID NO: 106 - RNA, 48 nt, Homo sapiens
mumamamgmamgmumamcmumgmamgmgmamcmumgmcmcmgmcmcmu

SEQ ID NO: 107 - RNA, 36 nt, Homo sapiens
mamamamamumamamgmamgmcmamcmcmcmgmcmu

SEQ ID NO: 108 - RNA, 44 nt, Homo sapiens
mamamamamumamamgmamgmcmamcmcmcmgmcmumumcmumc

SEQ ID NO: 109 - RNA, 48 nt, Homo sapiens
mamamamamumamamgmamgmcmamcmcmcmgmcmumumcmumcmumc

SEQ ID NO: 110 - RNA, 44 nt, Homo sapiens
mamamamamamamumamamgmamgmcmamcmcmcmgmcmumumc

SEQ ID NO: 111 - RNA, 36 nt, Homo sapiens
mamamumamamgmamgmcmamcmcmcmgmcmumumc

SEQ ID NO: 112 - RNA, 44 nt, Homo sapiens
mamamumamamgmamgmcmamcmcmcmgmcmumumcmumcmumc

SEQ ID NO: 113 - RNA, 44 nt, Homo sapiens
mamamamamumamamgmamgmcmamcmcmcmgmcmumumcmcmg

SEQ ID NO: 114 - RNA, 40 nt, Homo sapiens
mamamumamamgmamgmcmamcmcmcmgmcmumumcmumc

SEQ ID NO: 115 - RNA, 44 nt, Homo sapiens
mamamcmumamumamcmamamcmcmumamcmumamcmcmumcma

SEQ ID NO: 116 - DNA, 23 bp, Homo sapiens
gaucaaguccaauaucgaudtdt

SEQ ID NO: 117 - DNA, 23 bp, Homo sapiens
gcgugucaaaacuaacguadtdt

SEQ ID NO: 118 - DNA, 23 bp, Homo sapiens
agacgacggcgaugaggaadtdt

SEQ ID NO: 119 - DNA, 22 bp, Homo sapiens
cttaatgtcacgcacgatttcc

SEQ ID NO: 120 - DNA, 20 bp, Homo sapiens
aaatctggcaccacaccttc

SEQ ID NO: 121 - DNA, 20 bp, Homo sapiens
agaggcgtacagggatagca

SEQ ID NO: 122 - DNA, 20 bp, Homo sapiens
tcttcctcgctgtgtgtcag

SEQ ID NO: 123 - DNA, 20 bp, Homo sapiens
ttctgtgcacaagggctatg

SEQ ID NO: 124 - DNA, 20 bp, Homo sapiens
atggcagatgctgctctctt

SEQ ID NO: 125 - DNA, 37 bp, Homo sapiens; identical to SEQ ID NO: 1

SEQ ID NO: 126 - DNA, 67 bp, Homo sapiens; identical to SEQ ID NO: 2

SEQ ID NO: 127 - DNA, 46 bp, Homo sapiens
aggttcgaaccctgctcgctgcggaagcgggtgctcttattttttt

SEQ ID NO: 128 - DNA, 51 bp, Homo sapiens
ggttcaaatccgggtgccccctctgtgctccggagttacctcgttttgttg

SEQ ID NO: 129 - DNA, 56 bp, Homo sapiens
tccgggttcgagtcccggcagagatggtcacctggcaggtgcctcttttattttga

SEQ ID NO: 130 - DNA, 57 bp, Homo sapiens
gggttcgaatcccatcctcgtcggctaaggaagtcctgtgctcagttttgtagcatc

SEQ ID NO: 131 - DNA, 56 bp, Homo sapiens
ctgggttcgaatcccagcggtgcctcaaccgagcgtccaagctctttccatttttg

SEQ ID NO: 132 - RNA, 20 nt, Homo sapiens
gaagcgggugcucuuauuuu

SEQ ID NO: 133 - DNA, 19 bp, Homo sapiens
cuucgccccgagaataaaa

SEQ ID NO: 134 - DNA, 11 bp, Homo sapiens
tggccgagcgg

SEQ ID NO: 135 - DNA, 11 bp, Homo sapiens
tggccgagtgg

SEQ ID NO: 136 - DNA, 11 bp, Homo sapiens
ggttcgaatcc

SEQ ID NO: 137 - DNA, 11 bp, Homo sapiens
ggttcgaaccc

SEQ ID NO: 138 - DNA, 488 bp, Homo sapiens
ctcgcagaaatgccaaattttcgggagcgcgggctcgcagcacgtaacagaacagatctttttgtttcacggccctctcgtagacctaataagcgcgagggcgaaaaccaggtcagcgaccagaagccaggcaaggttgcgattttcggagcacagcgaaggggagagtttctaagttctgaatgtacctctatttagggtggtttctattatttcttcgcgtctaccctgtagttgccgcaccctgagccctcagccccgccgcttcgcacggagcggagctcagagggtgcgcgctccgccctttcgcgggcctggcatgagcgcagtggttgttacactaaagtgtctccgcctgtcgaatattctcgtggcagcgatggccgagtggttaaggcgttggacttgaaatccaatggggtctccccgcgcaggttcgaaccctgctcgctgcggaagcgggtgctcttatttttttctattttttt

SEQ ID NO: 139 - DNA, 482 bp, Homo sapiens
ctcgcagaaatgccaaattttcgggagcgcgggctcgcagcacgtaacagaacagatctttttgtttcacggccctctcgtagacctaataagcgcgagggcgaaaaccaggtcagcgaccagaagccaggcaaggttgcgattttcggagcacagcgaaggggagagtttctaagttctgaatgtacctctatttagggtggtttctattatttcttcgcgtctaccctgtagttgccgcaccctgagccctcagccccgccgcttcgcacggagcggagctcagagggtgcgcgctccgccctttcgcgggcctggcatgagcgcagtggttgttacactaaagtgtctccgcctgtcgaatattctcgtggcagcgatggccgagtggttaaggcgttggacttgaaatccaatggggtctccccgcgcaggttcgaaccctgcgtcttcgcttcactgaagaccattttttttctaga

(19) United States
(12) Patent Application Publication
(10) Pub. No.: US 2012/0110686 A1
(43) Pub. Date: May 3, 2012

(54) Cand45 tRNA-Derived Expression System for Gene Modulation

(76) Inventors: Mark A. Kay, Los Altos, CA (US); Dirk Haussecker, Buehlertal (DE)

(21) Appl. No.: 13/265,429

(22) PCT Filed: Apr. 19, 2010
(86) PCT No.: PCT/US10/31593

§ 371 Date: Jan. 4, 2012

Related U.S. Application Data
(62) Division of application No. 61/170,950, filed on Apr. 20, 2009.
Application No. 61/170,950 is a division of application No. 61/301,322, filed on Feb. 4, 2010.
Application No. 61/301,322 is a division of application No. 61/312,456, filed on Mar. 10, 2010.

(51) Int. Cl. C12N 15/63; C12P 19/34; A01K 67/027; C12N 1/21; C12N 5/10; C12N 5/071; C12N 1/19

(52) U.S. Cl. 800/14; 435/375; 435/320.1; 435/91.51; 435/252.3; 435/254.2; 435/419; 435/348; 435/325; 800/18

(57) ABSTRACT

The present invention relates to systems and methods for modulating gene expression and applications thereof. Provided is a novel expression system to generate RNaseZ and RNA polymerase III dependent RNAs to regulate genes and control the timing and the location of the regulation by supplying synthetic or expressed oligonucleotide antisense to a small RNA.

### CROSS REFERENCE TO RELATED APPLICATIONS

This application claims the benefit of, and priority to, U.S. Provisional Patent Application Ser. Nos. 61/170,950 filed Apr. 20, 2009, 61/301,322 filed Feb. 4, 2010, and No. 61/312,456, filed Mar. 10, 2010, the entire disclosures of which are hereby incorporated by reference in their entireties.

### FIELD OF THE INVENTION

The present invention relates to systems and methods for modulating gene expression through RNA interference (“RNAi”) and applications thereof.

### BACKGROUND OF THE INVENTION

RNA-based therapeutics are one of the promising areas for new drug development. Specifically, RNA interference (RNAi) provides a novel approach to treat human diseases. RNAi is an RNA-dependent gene silencing process that is mediated by the RNA-induced silencing complex (RISC) and is initiated by short double-stranded RNA (dsRNA) molecules in the cytoplasm, where they interact with the catalytic RISC component Argonaute, particularly Argonaute 2 in humans. When the dsRNA is exogenous, such as from an infection by a virus with an RNA genome or from laboratory manipulations, the RNA is imported directly into the cytoplasm and cleaved by Dicer into short fragments of dsRNA called small interfering RNAs (siRNAs). A synthetic siRNA may also be directly provided which then feeds into the RNAi pathway without the need for prior Dicer processing. The initiating dsRNA can also be endogenous, as in pre-microRNAs expressed from RNA-coding genes in the genome. The primary transcripts from such genes are first processed to the characteristic stem-loop structure of pre-microRNA in the nucleus, and then exported to the cytoplasm to be cleaved by Dicer to produce small silencing microRNAs which are analogous in structure and function to the siRNA. Thus, the two pathways for exogenous and endogenous dsRNA converge at the RISC complex, which mediates gene silencing effects.

siRNA, also known as short interfering RNA or silencing RNA, generally are 18-25 nucleotide-long dsRNA molecules, with preferably 2-nucleotide 3′-overhangs on each end. Each strand generally has a 5′-phosphate group and a 3′-hydroxyl (—OH) group. Typically, the siRNA is provided in a form that is 5′-hydroxylated and converted into the active 5′ phosphate form following delivery into cells.

There are several approaches to explore RNA interference to develop nucleic acid-based therapeutics as well as its use in gene discovery and target validation. However, there is still a need for alternative approaches to better control the timing and location of siRNA delivery.

### SUMMARY OF THE INVENTION

The present invention provides a method to inhibit the expression of a target gene in a cell. The method comprises: (a) introducing a first single stranded RNA into a cell, the first single stranded RNA comprising a ribonucleotide sequence which is antisense to and complementary to a nucleotide sequence of a target gene. In some embodiments, the first single stranded RNA also comprises a 5′-phosphate and/or at least 2 continuous uracils at the 3′-end. In step (b), separately introducing into the cell a second single stranded RNA comprising a ribonucleotide sequence complementary to a portion of the ribonucleotide sequence of the first single stranded RNA. In step (c), forming a double stranded RNA comprising the first single stranded RNA and the second single stranded RNA. The double stranded RNA substantially increases the ability of the first single stranded RNA to inhibit the expression of the target gene.

In various embodiments, the first single stranded RNA is transcribed from a DNA comprising an RNA polymerase III initiation site. In some embodiments, the DNA comprises an RNase recognition site. In some embodiments, the RNase is RNase Z. In some embodiments, the DNA comprises an RNase polymerase III terminator. In some embodiments, the first single stranded RNA is transcribed from a DNA by an RNA polymerase III. In some embodiments, the 5′-phosphate of the first single stranded RNA is generated by the cleavage of an RNase. In some embodiments, the first single stranded RNA has a length from 18 to 25 nucleotide. In some embodiments, the first single stranded RNA comprises 3 to 6 continuous uracils at the 3′ end. In some embodiments, the second single stranded RNA has a length of 14 to 25 nucleotides.

In some embodiments, the single stranded RNA does not possess substantial gene silencing activity without forming the double stranded RNA with the second single stranded RNA. In some embodiments, the double stranded RNA inhibits expression of the target gene through the RNA interference pathway.

The present invention also provides an expression vector comprising: (a) a first DNA sequence comprising an RNA polymerase III initiation site; (b) a second DNA sequence located downstream of the first DNA sequence, the second DNA sequence comprising an RNase recognition site; and (c) a third DNA sequence located downstream of the second DNA sequence, the third DNA comprising an RNA polymerase III terminator.

In some embodiments, the expression vector further comprises a fourth DNA sequence corresponding to the nucleotide sequence of a target gene. The fourth DNA sequence is located between the second DNA sequence and the third DNA sequence.

The present invention provides host cells derived from a cell transfected with the expression vector described herein, as well as animals comprising the host cells described herein.

The present invention provides a method of generating a single stranded RNA against a target gene. The method comprises: (1) transcribing a parent RNA from an expression vector with an RNA polymerase III; and (2) cleaving the parent RNA with an RNase Z to generate a single stranded RNA having a length from about 18 to about 25 nucleotides. The single stranded RNA comprises a 5′-phosphate and is antisense to and complementary to a portion of the nucleotide sequence of a target gene, wherein the target gene does not encode a tRNA.

### BRIEF DESCRIPTION OF THE DRAWINGS

FIG. 1 depicts an exemplary embodiment of the invention.

FIG. 2A-2C: Cand45 tsRNA is a 5′-phosphorylated, 3′ hydroxylated small RNA generated by RNaseZ processing. A. tsRNAs are 5′ phosphorylated and 3′ hydroxylated (Northern blot of diagnostic enzyme treatment). 293 cell RNA was treated with the following enzymes (potential activities described in parenthesis), and enzyme susceptibility of the 21-22 nt small RNAs of interest deduced by their shift in gel mobility or their disappearance in Northern blot: 1. buffer; 2. T4 polynucleotide kinase (PNK)+ATP (5′ phosphorylation of 5′ hydroxyl and 3′ dephosphorylation); 3. T4 PNK, then Terminator (removes 5′ monophosphorylated RNAs; Terminator-dependent RNA removal would indicate 5′OH RNAs); 4. Terminator; 5. T4 RNA ligase +ATP (for 5′P-3′OH RNAs: intramolecular circularization and/or trans-ligation to RNA containing either of these modifications); 6. Tobacco acid phosphatase (TAP; hydrolyzes phosphoric acid anhydride bonds in triphosphate of RNA caps, leaving 5′ monophosphate), then T4 RNA ligase (TAP-dependent T4 RNA ligation would indicate 5′ cap); 7. TAP (hydrolyzes phosphoric acid anhydride bonds in triphosphate of RNA caps, leaving 5′ monophosphate); 8. T4 RNA ligase, no ATP+3′ adapter oligo (adapter ligation would indicate 3′0H); 9. T4 PNK, then T4 RNA ligase, no ATP+3′ adapter oligo (PNK-dependent adapter ligation would indicate either 3′P, or 2′-3′ cyclic P); 10. 3′ exonuclease-negative T4 PNK, then T4 RNA ligase, no ATP+3′ adapter oligo (would confirm that a reaction in treatment ‘9’ was dependent on 3′ de-phosphorylation by T4 PNK); 11. polyA polymerase (PAP; adds polyA to 3′ hydroxyl RNAs). Blots were stripped and re-hybridized; arrow indicates ˜20-22 nucleotide RNA of interest. B. Cand45 expression is unchanged in a HCT116-derived cell line that contains a mutation in the Dicer helicase domain. Most (e.g. miR-20 and miR-21), but not all (e.g. let-7a) microRNAs are down-regulated in this cell lineWt: parental HCT Dicer wild-type cell line; Dcr mut: HCT-derived Dicer helicase mutant cells; T4: test for T4 RNA ligase sensitivity (−: untreated; +: treated). C. RNaseZ/P processing of cand45 tRNA in vitro. A radiolabelled, in vitro transcribed cand45 precursor tRNA was treated with buffer alone (‘mock’), recombinant human RNaseZ and/or purified human RNaseP. Arrows indicate that RNA was treated sequentially with stated conditions. Reaction products (schematic for predicted fragments shown on the right) were visualized on a polyacrylamide gel. M: Decade (Ambion) RNA size marker.

FIG. 3: tsRNAs localize to the cytoplasm (Northern blot analysis of nuclear-cytoplasmic RNA fractionation). Sno38b and U6 snRNA serve as nuclear markers. Equal amounts of nuclear and cytoplasmic RNA were loaded, and blots stripped and re-hybridized. N: nuclear RNA fraction; C: cytoplasmic RNA fraction; arrow: T4 RNA ligase (T4)-sensitive RNA of interest (−: untreated; +: treated); M: Decade (Ambion) RNA size marker.

FIG. 4: tsRNAs associate with human Argonautes 1-4, but not Mov10. A. FLAG-Argonautes and FLAG-Mov10 were expressed at similar levels in 293 cells (Western blot); actin used for normalization. B+C. Northern blot analysis of RNA co-immunoprecipitations with FLAG epitope-tagged Gfp (negative control), human Argonautes 1-4 (A1-4), Mov10 (M10), and either HDAg (B), or TRBP (C). Blots were stripped and re-hybridized; B and C. show results from independent immunoprecipitations. Input: RNA isolated from 10% lysate used per immunoprecipitation; IP: immunoprecipitated RNA.

FIGS. 5A, 5B: Investigation of cand45-like small RNAs. A. Selected cand45-like candidate sequences (in red) with predicted RNaseZ cleavage sites (‘Z’). Shown are the sequences of the tRNA 3′ ends and RNA polymerase III termination region. B. Cand45-like small RNA candidate expression analysis (Northern blot). Cand45-like small RNAs can be detected as discrete 21-28 nucleotide small RNAs and are modulated by Argonaute overexpression. Gfp (negative control), Dicer, Ago 1-4, Mov10: transfected FLAG epitope-tagged expression plasmids. Terminator treatment (‘TER’; −: untreated; +: treated) was used to determine the 5′ phosphorylation status of cand45-like small RNA candidates. In these blots, the amount of +/−Terminator-treated Argonaute4 RNA loaded was half that of the other samples. M: Decade (Ambion) RNA size marker.

FIG. 6A-6E: Trans-silencing capacity of tsRNAs (dual luciferase assay). A. (B) Cand45 overexpression from a plasmid into which the genomic sequence of cand45 had been cloned (cand45-45; by Northern blot). Cand45-empty: cand45 cloning plasmid with only a cloning site between the cand45 RNaseZ cleavage site and the RNA PolIII terminator; cand45-con: cand45-derived expression plasmid in which the cand45 sequence in cand45-45 was replaced with an arbitrary control sequence; M: Decade (Ambion) RNA size marker. B. Cand45 overexpression-dependent, anti-cand45 antisense-induced trans-silencing in HCT116 cells. Dual luciferase assay with reporter gene carrying a fully cand45-complementary target site in its 3′ UTR of Renilla luciferase (psi-cand45). Cand45-empty, cand45-con, cand45-45 as in ‘A’. C. Confirmation of specificity of the anti-cand45 induced trans-silencing effect by the use of three (antisense) control oligonucleotides. D. Anti-cand45 induced trans-silencing is RNAi-related. Trans-silencing is enhanced by overexpression of slicing-competent Argonaute 2, but mitigated by the non-slicing Argonautes 1, 3, and 4. E. Predicted cand45:anti-cand45 duplex; A.U.: arbitrary units; error bars indicate standard deviation from n=3 transfections.

FIG. 7A-7C: SITS silencing is due to preferential Ago2-RiSC incorporation of reconstituted dsRNA. A. Cand45 Argonaute 2 co-immunoprecipitation efficiency is enhanced by addition of complementary oligonucleotide (quantitation of Northern blots from Ago co-IP experiments). B. Cand45-+2 Argonaute 2 co-immunoprecipitation efficiency is enhanced by addition of complementary oligonucleotide. Schematic of predicted duplex structures shown in ‘A’ and ‘B’. C. Structure-based Argonaute incorporation of small RNAs (model).

FIG. 8 Structure-function analysis of SITS (dual luciferase assays). Four different vectors for the expression of RNA 1 (guide RNA) which differ in their lengths were combined with various complementary synthetic RNA 2 and the effect on silencing of Renilla luciferase containing a complementary target site measured. ‘100’: no silencing; ‘0’: complete silencing.

FIG. 9: General applicability of SITS (dual luciferase SITS silencing assay with cand45 tsRNA expression vector and complementary oligonucleotides with various modifications). SITS is amenable to nucleotide modifications (e.g. at least 40% 2′-O-methyl or LNA) and works in various cell lines (here: mouse embryonic fibroblasts, MEF).

FIG. 10A-10C: Depiction of the CAND45 derived expression system. A. The core tRNA promoter elements, Box A and Box B. B. Depiction of the genomic cand45 sequences used in the CAND45 derived expression system. C. One embodiment of the invention, where part of the cand45 genome sequence is cloned to construct an expression vector.

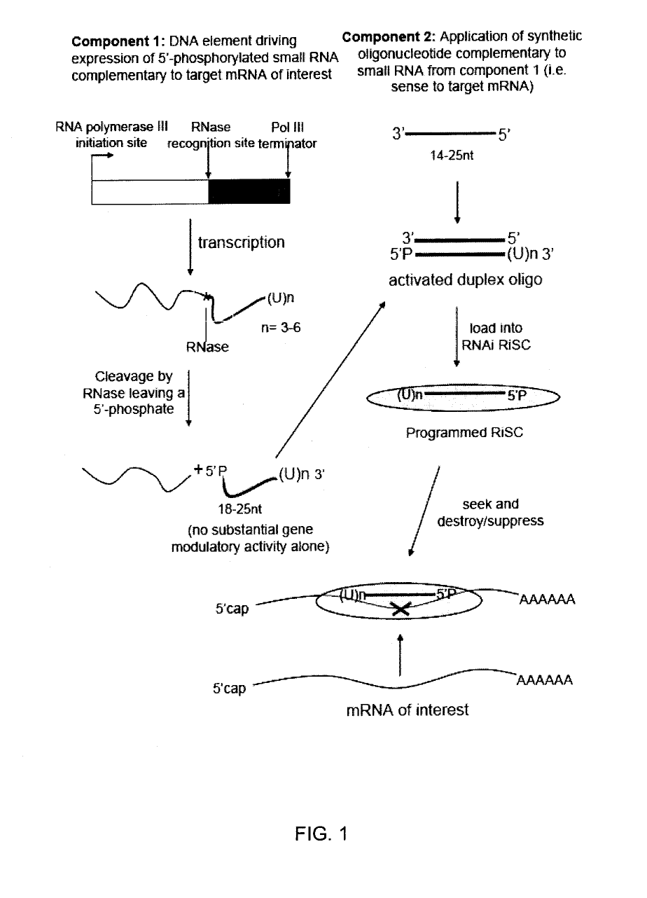

### DETAILED DESCRIPTION OF THE INVENTION

The present invention provides a novel system to induce gene silencing by providing an oligonucleotide of sense polarity with respect to the target gene. Generally, gene silencing or gene knockdown is induced by the addition of either double stranded RNAs or oligonucleotides antisense to the target gene. The system of the present invention is different from other gene knockdown systems. In the present system the addition of an oligonucleotide sense to the target gene induces the gene silencing.

### I. Overview

RNAi and related pathways are inherently potent, natural pathways for the down-regulation or up-regulation of genes with sequence complementarity in trans. One obstacle to harnessing this pathway is the delivery of the typically double stranded RNA into cells, particularly in living animals in vivo. A number of single stranded oligonucleotides (e.g. phosphorothioate oligos) on the other hand cross cell membranes more easily compared to double stranded nucleic acids. However, their inherent gene silencing potential is generally limited compared to RNAi. RNAi can also be induced by non-synthetic means, e.g., using viral delivery in a gene therapy-type of approach to express double stranded RNAs from a DNA vector. However, one significant safety problem is that most of these gene therapy systems cannot easily be regulated in a temporal manner. The present invention provides a system taking advantage of the relative ease of administering single stranded oligonucleotides to organs such as the liver, spleen, and the CNS to tap into the potent RNAi-related cellular gene silencing mechanisms. For example, an oligonucleotide of sense polarity to the target gene is administered to cells containing RNAs derived from and channeled into the cand45 pathway, such as from a cand45-derived expression vector described herein. This activates the cand45 pathway for temporally regulated, easily administered RNAi-related gene silencing.

In eukaryotes, including mammals, tRNAs are encoded by families of genes that are 73 to 150 base pairs long. A tRNA gene is transcribed by DNA-dependent RNA polymerase III (also called Pol III) into a tRNA precursor (pre-tRNA), a tRNA that essentially has additional sequences at its 5′ and 3′ end that must be removed for it to become a biologically active tRNA in protein translation. These additional sequences are referred to as 5′ leader and 3′ trailer. The maturation of the pre-tRNA to tRNA depends on certain conserved sequences and secondary structures of the molecule and at least two enzyme systems that are present in every cell, such as RNase P (Altman S. (2000) Nat Struct Biol 7, 827) and the 3′ processing enzyme RNase Z (Schiffer et al. (2002) EMBO J. 21, 2769; Pellegrini et al. (2003) EMBO J; 22, 4534).

Research by the inventors on RNAi-related, human tRNA-derived small RNAs in human cells led to the surprising discovery that providing an oligonucleotide of sense polarity to a target gene can induces RNA silencing in trans in a cell when a small RNA of antisense polarity to the target gene is present in the cell. This is unexpected because the expressed antisense small RNA by itself has no or very limited silencing activity even though it is associated with RNAi effector proteins. Similarly, the introduction of single-stranded guide RNAs, or the temporally separate introduction of guide and passenger strands do not efficiently induce RNA silencing.

Inducing RNA silencing by providing a sense oligonucleotide is not only a novel way of inducing gene silencing, it is also particularly valuable for knocking down genes in vivo (e.g., in gene function studies and therapeutics). Accordingly, it is now possible, e.g., to combine the relative ease and simplicity of delivering unformulated single-stranded oligonucleotides to organs like the liver, spleen, lung, intestine, heart, eye, and the CNS to, as well as non-normal tissues such as tumors in a temporally regulated manner, tap into the inherently more potent RNAi-related mechanisms once inside cells in a temporally regulated fashion.

### II. Method of Gene Silencing

In one aspect, the present invention provides a method for gene silencing. The method comprises: (a) introducing a first single stranded RNA into a cell, the first single stranded RNA comprising a ribonucleotide sequence which is antisense to and complementary to a nucleotide sequence of a target gene; (b) separately introducing into the cell a second single stranded RNA comprising a ribonucleotide sequence complementary to a portion of the ribonucleotide sequence of the first single stranded RNA; and (c) forming a double stranded RNA comprising the first single stranded RNA and the second single stranded RNA, the double stranded RNA inhibits the expression of the target gene. The first single stranded RNA can be introduced before, or after, the introduction of the second single stranded RNA.

In some embodiments, the first single stranded RNA comprises a 5′-phosphate and at least 2 continuous uracils at the 3′-end.

### A. Gene Silencing and Target Gene

The method of the invention generally relates to gene silencing of a target gene with an RNA silencing agent,

“Gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi). “RNA interference” refers to siRNA-mediated cleavage of target mRNA through a pathway whereby small inhibitory RNA (siRNA) acts in concert with host proteins (e.g., the RNA induced silencing complex, RISC) to degrade messenger RNA (mRNA) in a sequence-dependent fashion.

The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has, e.g., fluorescent properties (e.g., GFP) or enzymatic activity (e.g., alkaline phosphatases), or several other procedures.

“Gene knockdown” refers to the techniques by which the expression of one or more of an organism's genes (including non-coding genes) is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knockdown organism”. If the change in gene expression is caused by an oligonucleotide binding to an mRNA or temporarily binding to a gene, this results in a temporary change in gene expression without modification of the chromosomal DNA and is referred to as a “transient knockdown”.

In a transient knockdown, the binding of the oligonucleotide to the active gene or its transcripts causes decreased expression through blocking of transcription (in the case of gene-binding), degradation of the mRNA transcript (e.g. by siRNA or RNase H dependent antisense) or blocking either mRNA translation, pre-mRNA splicing sites or nuclease cleavage sites used for maturation of other functional RNAs such as miRNA (e.g. by Morpholino oligos or other RNase H independent antisense).

“Target” or “target gene” refers to a gene whose expression is selectively inhibited or “silenced.” This silencing is achieved by cleaving or translationally silencing or enhance RNA degradation of the mRNA of the target gene (also referred to herein as the “target mRNA”) by an siRNA, microRNA, or an RNA silencing agent, e.g., an siRNA synthesized enzymaticaly or non-enzymatically, or created from an engineered RNA precursor by a cell's RNA silencing system.

“RNA silencing agent” refers to an RNA (or analog thereof), having sufficient sequence complementarity to a target RNA (i.e., the RNA being degraded) to direct RNA silencing (e.g., RNAi). An RNA silencing agent having a “sequence sufficiently complementary to a target RNA sequence to direct RNA silencing” means that the RNA silencing agent has a sequence sufficient to trigger the destruction or post-transcriptional silencing of the target RNA by the RNA silencing machinery (e.g., the RISC) or related process. An RNA silencing agent having a “sequence sufficiently complementary to a target RNA sequence to direct RNA silencing” also means that the RNA silencing agent has a sequence sufficient to trigger the translational inhibition of the target RNA by the RNA silencing machinery or process.

An RNA silencing agent can be a single stranded RNA or an siRNA, which may be double stranded, or single stranded but can form a duplex structure as described herein.

As outlined herein, “siRNA” means small inhibitory RNA duplexes that induce the RNA interference (RNAi) pathway. These molecules can vary in length (generally 18-30 base pairs) and contain varying degrees of complementarity to their target mRNA in the antisense strand, also known in the art as the guide strand. Some, but not all, siRNA have unpaired overhanging bases on the 5′ or 3′ end of the sense strand and/or the antisense strand. The term “siRNA” includes duplexes of two separate strands, which in some cases may also be derived from single strands that can form hairpin structures comprising a duplex region.

The term “ribonucleotide” or “ribonucleic acid (RNA)” refers to a modified or unmodified nucleotide or polynucleotide comprising at least one ribonucleotide unit. A ribonucleotide unit comprises a hydroxyl group attached to the 2′-position of a ribosyl moiety that has a nitrogenous base attached in N-glycosidic linkage at the 1′-position of a ribosyl moiety, and a moiety that either allows for linkage to another nucleotide or precludes linkage.

A “nucleotide” is a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates, locked nucleic acid, unlocked nucleic acids, and peptides.

Modified bases include nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term “nucleotide” also includes what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′-oxygen with an amine group.

An “antisense” or “antisense strand” of an siRNA or RNA silencing agent is a strand that is substantially complementary to a section of about 10-50 nucleotides, e.g., about 15-30, 14-25, 18-23 or 19-22 nucleotides of the mRNA of the gene targeted for silencing. The antisense strand has sequence sufficiently complementary to the desired target mRNA sequence to direct target-specific RNA silencing, (e.g., for RNAi, complementarity sufficient to trigger the destruction of the desired target mRNA by the RNAi machinery or related process).

A “sense strand” of an siRNA or RNAi agent is a strand that is complementary to the antisense strand.

“Duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may exist as 5′ and 3′ overhangs, for example. Further, within the duplex region, 100% complementarity is not required; substantial complementarity is allowable within a duplex region. Substantial complementarity refers to 79% or greater complementarity. For example, a mismatch in a duplex region consisting of 19 base pairs results in 94.7% complementarity, rendering the duplex region substantially complementary.

“Complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity.

### B. First Single Stranded RNA

In one aspect of the invention, a first single stranded RNA is introduced into the cell. The first single stranded RNA comprises a ribonucleotide sequence that is antisense to and complementary to a nucleotide sequence of a target gene. In some embodiments, the first single stranded RNA also comprises a 5′-phosphate and/or at least 2 continuous uracils at the 3′-end.

The first single stranded RNA has a length of at least 14 nucleotides, preferably more than 15, 16, 17, or 18 nucleotides. Exemplary length includes from about 18 to about 100, from about 18 to about 50, from about 20 to about 30 or from about 21 to about 28 nucleotides. In some embodiments, the first single stranded RNA has a length from about 18 to about 25 nucleotides. The sequence of the first single stranded RNA depends on the sequence of the target gene, i.e., the first single stranded RNA and the second single stranded RNA are capable of forming a duplex and acting through the RNAi pathway. In some embodiments, the sequence of the first single stranded RNA is designed using methods known in the art for designing siRNA, such as the siDESIGN® Center (Dharmacon) and the siRNA Target Designer (Promega).

In the present invention, within the definition of the first stranded RNA includes a polynucleotide of fully modified RNA, (e.g. LNA or 2′-O-methyl). Thus in some embodiments, the RNAs encompass a polynucleotide which every nucleotide is modified, which also including both cyclic or acyclic residues.

Generally, the sequence of the first single stranded RNA is designed to minimize or eliminate off-target silencing. “Off-target silencing” or “off-target interference” refers to the degradation of mRNA other than the intended target mRNA due to overlapping and/or partial homology with secondary mRNA messages.

In general, the 5′-end of the first single stranded RNA is phosphorylated; at least one phosphate group is attached to a chemical (e.g., organic) compound. Phosphate groups can be attached, for example, to proteins or to sugar moieties. “5′-phosphorylated” refers to polynucleotides or oligonucleotides having a phosphate group attached via an ester linkage to the C5-hydroxyl of the 5′-sugar (e.g., the 5′-ribose or deoxyribose, or an analog of the same). Mono-, di-, and tri-phosphates are common. Also included within the scope of the invention are phosphate group analogs that function in the same or similar manner as the mono-, di-, or tri-phosphate groups found in nature (see, e.g., exemplified analogs).

In general, the 5′-phosphate of the first single-stranded RNA is generated by the cleavage of an RNase, such as RNase Z as described herein.

“Ribonuclease” or “RNase” is a nuclease catalyzing the degradation of RNA into smaller components. Ribonucleases can be divided into endoribonucleases and exoribonucleases, and comprise several sub-classes within the EC 2.7 (for the phosphorolytic enzymes) and 3.1 (for the hydrolytic enzymes) classes of enzymes.

In general, the 3′-terminus of the first single stranded RNA has at least one uracil, preferably two or more uracils. There may be 2, 3, 4, 5, 6, 7, 8, 9, or more uracils. The uracils are preferably located at the very end of the RNA and preferably are continuous.

In general, the uracils at the 3′terminus of the first single stranded RNA are added by an RNA Polymerase, preferably a Pol III as described herein.

Pol III transcribes DNA to synthesize ribosomal 5S rRNA, tRNA and other small RNAs. Pol III generally requires no control sequences upstream of the gene (as opposed to Pol II). Pol III instead normally relies on internal control sequences—sequences within the transcribed section of the gene, although upstream sequences are occasionally seen. Pol III generally terminates transcription at small poly(T) stretch (5-6).

Generally, the first single stranded RNA is introduced into the cell by transcribing from a DNA template in the cell. In some embodiments, the DNA template is derived from an expression vector that comprises DNA encoding the first single stranded RNA as described herein that is introduced into the cell using methods known in the art.

“A DNA template is derived from an expression vector” is meant a DNA template, or a part it, the origin of it can be traced back to the expression vector. For example, after an expression vector is transfected into a cell, the expression vector may not remain intact over time. Part of the expression vector sequence may be lost with the passage of the cells. However, the fragment encoding the exogenous gene (the insert) may stay in the cells for generations and serve as a template for transcription. Thus, the DNA template is derived from the expression vector even though part of the original vector sequences is lost over time.

Generally, a pre-RNA is transcribed from DNA by Pol III. Pol III transcription normally terminates at a Pol III terminator. “Pol III terminator” is a stretch of thymines, such as 4 to 6 thymines. Variations with limited non-thymine residues are also known and included in our definition. The termination by Pol III therefore generates a pre-RNA with two or more continuous uracils at the 3′-end, such as 2, 3, or more. All tRNA molecules are transcribed as long precursors with extra sequences at their 5′ and 3′ termini. Hence, the precursor tRNAs (pre-tRNAs) undergo several processing steps, including the removal of the 5′- and 3′-extra sequences, to generate mature tRNAs. Several ribonucleases are involved in the 5′- and 3′-processing. The removal of the 5′-extra sequence is accomplished by RNase P, a ribonucleoprotein. In eukaryotes, the tRNA genes do not encode the 3′-terminal CCA sequence, which is essential for aminoacylation. The eukaryotic 3′-processing is done mainly by a tRNA 3′-processing endoribonuclease named tRNase Z (EC 3.1.26.11; RNase Z or 3′-tRNase), which cuts the pre-tRNAs with the 3′-extra sequence after the discriminator base. Then the CCA sequence is added by tRNA nucleotidyltransferase. See Ishii et al., J. Biol. Chem. (2005)280:14138-14144, herein incorporated by reference.

In some embodiments, the first single stranded RNA is transcribed from a DNA comprising a Pol III initiation site. The sequence of the Pol III initiation site generally depends on the tRNA and generally is 17-19 bp upstream of the intragenic sequence element ‘BoxA’ which is well known in the art.

In some embodiments, the nucleic acid constructs of the present invention incorporate a Pol III promoter. The nucleic acid constructs incorporate a “box A” and a “box B” which comprise the Pol III promoter sequence. By constructing the nucleic acid constructs with an independent promoter, the genes can be transcribed independently of a separate plasmid promoter; therefore, the unregulated promoter should express constitutively in all animal cells or tissues. Further, Pol III promoters tend to be more or less universal in their expression and should function equally well in a wide range of host cell systems. Also, such promoters do not appear to have “enhancer” activity which are potentially carcinogenic.

The actual promoter sequence could have different embodiments, and is not limited herein by the previous description. For example, the promoter region including the upstream region, the transcription initiating region, “Box A” and “Box B” can be taken directly from any highly active, natural tRNA. For example, a tRNA promoter sequence which has been shown to be particularly strong is the Glu tRNA gene, in mouse. The Glu tRNA gene has the advantage that it is straightforward to use as an active promoter and the short tRNA sequence which will be transcribed should not have any effect on the activity of the nucleic acid construct.

In some embodiments, the DNA comprises an RNase recognition site. In some embodiments, the RNase is RNase Z. It has been suggested that RNaseZ minimal substrates is a 7 bp stem (corresponding to the acceptor stem of a tRNA) with adjacent T stem-loop-like structure. Cleavage occurs one nucleotide downstream of the double-stranded region (corresponding to the discriminator base in a tRNA) on the side with the T stem-loop structure. Nashimoto et al. NAR 26: 2565-2572 (1998).

In some embodiments, the 5′-phosphate of the first single stranded RNA is generated by the cleavage by an RNase, such as RNaseZ.

Generally, the single stranded RNA does not possess substantial gene silencing activity without forming the double stranded RNA with a second single stranded RNA as described herein.

“Substantial gene silencing activity” refers to the ability of an RNAi agent to down-regulate the expression of a target gene, as measured either at the mRNA level or protein level, by at least about 10%, 20%, 30%, 40%, 50%, 60%, 70%, 80%, 90% or 100% in a target cell of interest, or by at least about 10%, 20%, 30%, 40%, 50%, 60%, 70%, 80%, 90% or 100% in a population of cells of interest.

In general, RNAi in a cell is carried out by the introduction into the cell of a double stranded RNA or a single stranded RNA that can self-fold to form a duplex structure. The double stranded RNA or single stranded RNA are generated either in vitro by chemical or enzymatic synthesis, or in vitro by transcription from a DNA template encoding the siRNA or precursor. In traditional methods both strands of the siRNA (sense and antisense), either as two molecules or as one single molecules, are introduced into the cell concurrently to activate the RNAi pathway. In contrast, the present invention provides for the introduction of the two RNA strands separately. The introduction of one of the single stranded RNAs does not activate the RNAi pathway. The RNAi pathway is only activated only after both single stranded RNAs are introduced into the cell. Therefore, the single stranded RNA, particularly the first single stranded RNA, does not possess substantial gene silencing activity without forming the double-stranded RNA with a second single stranded RNA.

### C. Second Single Stranded RNA

In another aspect of the invention, a second single stranded RNA is separately introduced into the cell. The second single stranded RNA comprises a ribonucleotide sequence complementary to a portion of the ribonucleotide sequence of the first single stranded RNA.

The second single stranded RNA has a length of at least about 12 nucleotides, preferably more than about 15, 16, 17, or 18 nucleotides, such as from about 18 to about 100, about 18 to about 50, about 20 to about 30 or about 21 to about 28 nucleotides. In some embodiments, the second single stranded RNA has a length from about 18 to about 25 nucleotides. The sequence of the second single stranded RNA also depends on the sequence of the target gene. As described herein, the first single stranded RNA and the second single stranded RNA form a duplex and act through the RNAi pathway. Thus, after the sequence of the first single stranded RNA is determined, the second single stranded RNA includes a sequence that is complementary to the sequence of the first single stranded RNA.

The complementary portion of the first single stranded RNA and the second single stranded RNA varies, as long as it provides sufficient stability and enables the two single stranded RNA molecules to form a double stranded RNA capable of silencing the target gene. The length of the complementary portion is preferably at least about 14 nucleotides, more preferably about 15, 16, 17, 18, 19, 20, 21, 22, 23, 24, or 25 nucleotides or more.

The second single stranded RNA may also have a sequence that is not complementary to the first single stranded RNA as long as the two RNAs are able to form a double stranded RNA to activate the RNAi pathway.

The second single stranded RNA is generated and introduced into the cells using methods known in the art.

In some embodiments, the second single stranded RNA is chemically synthesized. See US Patent Application Publication No. 2009/0005549, incorporated herein by reference in its entirety. In some embodiments, the second single stranded RNA comprises a modification at one or more of the nucleosides as described herein and as known in the art.

In some embodiments, the second single stranded RNA is generated enzymatically in vitro using methods known in the art.

In some embodiments, the second single stranded RNA is generated in vivo by transcription from a DNA using methods known in the art. Generally, the DNA is a DNA fragment or an expression vector transfected into the cell.

When the second single stranded RNA is generated in vivo, it is optionally introduced into the cells using methods known in the art, including, but not limited to, liposomes and nanoparticles, such as, the lipid nanoparticle described in US Application Publication No. 2009/0048197, incorporated herein by reference in its entirety.

The first single stranded RNA can be introduced into the cells first and the second single stranded RNA is introduced subsequently, or vice versa.

The time lapse between the introduction of the two single stranded RNAs varies, such as from hours to days, weeks, or months, as appropriate for the purpose of gene silencing.

After both the first single stranded RNA and the second single stranded RNA are introduced into the cells, the two single stranded RNAs form a double stranded RNA comprising the first single stranded RNA and the second single stranded RNA. The double stranded RNA inhibits the expression of the target gene. In some embodiments, the double stranded RNA inhibits expression of the target gene through the RNA interference pathway.

The structure of the double stranded RNA formed by the first single stranded RNA and the second single stranded RNA varies. It can have one blunt end and one overhang, two blunt ends or two overhangs. The overhangs can be a 3′-end overhang or a 5′-end overhang. The length of the overhang is generally from about 1 to about 5 nucleotides (e.g., 1, 2, 3, 4, 5 or more), though a longer overhang is used in some embodiments.

“Overhang” or “nucleotide overhang” refers to the unpaired nucleotide or nucleotides that protrude from the duplex structure of a dsRNA when a 3′-end of one strand of the dsRNA extends beyond the 5′-end of the other strand, or 5′-end extending beyond a 3′-end.

A “blunt” or “blunt end” is a terminus of a dsRNA having no unpaired nucleotides at the end of the dsRNA, i.e., no nucleotide overhang. A “blunt ended” dsRNA is generally a dsRNA that is double stranded over its entire length, i.e., no nucleotide overhang at either end of the molecule. Some blunted dsRNA, however, may have internal mis-matches.

In some embodiments, the duplex has at least one blunt end, e.g., the 3′-end of the second single stranded RNA and the 5′-end of the first single stranded RNA forms a blunt end. However, in some embodiments, an overhang is preferred. For example, the 3′-end of the second single stranded RNA have about 1 to about 5 (e.g. 1, 2, 3, 4, or 5) more or less nucleotides than the 5′-end of the first single stranded RNA to form an overhang. Generally, the 3′-end of the first single stranded RNA has one or more uracils, thus it is longer than the 5′-end of the second single stranded RNA, and therefore forms an overhang. However, in some embodiments, the 5′-end of the second single stranded RNA is designed to have one or more adenines and optionally other additional nucleotides, thus forming a blunt end or an overhang with the 5′-end of the second single stranded RNA protruding.

In some embodiments, the first single stranded RNA is first introduced into the cell, e.g., by transfecting an expression vector encoding the first single stranded RNA into the cell, either transiently or by establishing a stable cell line or a transgenic animal. The first single stranded RNA is transcribed from the expression vector, or a fragment of it. This is followed by the introduction of the second single stranded RNA, optionally delivered by a liposome or by a nanoparticle, or other methods known in the art.

Alternatively, the second single stranded RNA is also transcribed from an expression vector or a fragment thereof, and the expression vector encoding the second single stranded RNA is introduced into the cell by transfection, either transiently or by establishing a stable cell line or a transgenic animal.

In some embodiments, the first single stranded RNA and the second single stranded RNA are transcribed from a single expression vector, though temporally separated under the control of different control elements of the expression vector as described herein.

In some embodiments, the first and second single stranded RNAs form a duplex that is loaded into the RNAi effector RISC without prior processing by the Dicer enzyme. In other embodiments, the duplex formed by the first and second single stranded RNAs is first processed by Dicer into shorter duplexes before loading onto RISC. In the former case, such structures generally comprise duplexes up to a size of about 25 base pairs, in the latter case, such structures generally comprise duplexes of 24 base pairs or longer.

### III. Expression Vector

In another aspect, the present invention provides an expression vector comprising: (a) a first DNA sequence comprising a Pol III initiation site; (b) a second DNA sequence located downstream of the first DNA sequence, the second DNA sequence comprising an RNase recognition site; and (c) a third DNA sequence located downstream of the second DNA sequence, the third DNA comprising a Pol III terminator. Generally, the DNA sequences of the vector are operably linked.

“Vector” is a nucleic acid molecule used as a vehicle to transfer foreign genetic material into another cell. Vectors also can be synthesized. Vectors introduce nucleic acids into host cells, where it can be reproduced. Examples are, plasmids, cosmids, and bacterial and yeast artificial chromosomes. Vectors are often recombinant molecules containing nucleic acid sequences from several sources. Vectors include viruses, for example adenovirus, adeno-associated virus (AAV), or retrovirus.

“Deoxyribonucleotide” and “DNA” refer to a nucleotide or polynucleotide comprising at least one sugar moiety that has an H, rather than an OH, at its 2′ and/or 3′ position.

A “polyribonucleotide” is a polynucleotide comprising two or more modified or unmodified ribonucleotides and/or their analogs. The term “polyribonucleotide” is used interchangeably with the term “oligoribonucleotide.”

Mammalian siRNA expression vectors (e.g., pKD mammalian siRNA expression vectors) essentially use a Pol III promoter as found in genes coding for U6 and H1 RNAs that is placed upstream of the siRNA encoding sequences are available from Upstate Biotechnology, Inc. a subsidiary of Millipore Corp. In these vectors, the coding sequence for the first strand and the coding sequence for the second strand of the siRNA are separated not by a tRNA sequence but by an unrelated spacer sequence. Upon transcription an RNA hairpin structure is formed with the upper and lower siRNA strand base-pairing. This molecule is then processed by “cellular nuclease” into a functional siRNA. This principle was first described by Myagishi and Taira (2002) Nature Biotechnol 19:497-500 and later adapted by others (Reviewed by Tuschl (2002) Nature Biotechnol 20: 446-448). The short oligo-U sequence that is added at the 3′-end of an siRNA molecule due to the presence and partial transcription of the Pol III termination signal do not change the specificity of the siRNA molecule. A short loop connecting the first and second strand of the siRNA molecule, that will also be the product when exchanging a tRNA intron with the coding sequence for a siRNA, will be processed/cleaved by cellular enzymes, resulted in an active siRNA molecule.

U.S. Patent Application Publication No. 2005/0203047 discloses a tRNA vector system for the delivery and expressions of short interfering nucleic acid, siRNA and micro RNA (miRNA) and antisense (asRNA) into an organism, which is incorporated herein by reference. The vector has a tRNA gene and a sequence coding siRNA, miRNA or antisense RNA is fused to the structural tRNA gene. The system is used to express siRNA, miRNA or antisense RNA.

In some embodiments, the expression vector of the present invention does not contain a tRNA gene sequence.

In some embodiments, the expression vector of the present invention comprises a tRNA gene and the tRNA gene encodes a naturally occurring tRNA-derived small RNAs (tsRNA). In some embodiments, the tsRNA is cand45.

A “tRNA-derived small RNAs” is a small RNA derived from a tRNA gene that interacts, or has the potential to interact with components of the RNAi or related pathways.

In some embodiments, the expression vector further comprises a fourth DNA sequence corresponding to the nucleotide sequence of a target gene, the fourth DNA sequence located between the second DNA sequence and the third DNA sequence.

In some embodiments, the expression vector of the present invention comprises a tRNA gene and a DNA sequence with a portion that corresponds to the target gene and encodes a sense RNA and is operably linked to the 3′-end of the tRNA gene sequence upstream of the transcription stop. Preferably, the sense RNA is not a miRNA.

In some embodiments, the expression vector of the present invention comprises a tRNA gene and a DNA sequence that does not express siRNA, miRNA or antisense RNA.

In some embodiments, the expression vector comprises a promoter such as the expression of the first single stranded RNA can be controlled, e.g. using the tetracycline systems described in U.S. Pat. Nos. 5,464,758 and 6,133,027, or other suitable small molecule based systems known in the art.

In some embodiments, the expression vector further comprises a sequence encoding the second single stranded RNA, such that the two single stranded RNAs are expressed from the same expression vector, but are controlled under two different promoters.

The expression vectors of the present invention also comprise elements known in the art as essential to an expression vector. The expression vectors may contain transcriptional and translational regulatory sequences, including, but not limited to, promoter sequences, ribosomal binding sites, transcriptional start and stop sequences, translational start and stop sequences, transcription terminator signals, polyadenylation signals, and enhancer or activator sequences. In one embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

### CAND45 Derived Expression System

In another aspect, the present invention provides a cand45 derived expression system. This express system comprises a partial cand45 genomic sequence that includes the core promoter sequence.

The core tRNA promoter elements, box A and B are intragenic, i.e. contained within the mature tRNA sequence. In addition, many insertions/deletions in the mature tRNA sequence between and outside of box A and B normally reduce tRNA gene expression efficiency. In some embodiments of the present invention, in the cand45 vector system, the DNA sequence encoding a non-coding RNA is either inserted (a) downstream of the RNaseZ cleavage site, or (b) downstream of box B with removal of a functional RNaseZ cleavage site. When the DNA sequence encoding a non-coding RNA is inserted downstream of the RNaseZ cleavage site, the RNA expressed from the vector has a 5′ phosphate. When the DNA sequence encoding a non-coding RNA is inserted downstream of box B with removal of the RNaseZ cleavage site, the RNA expressed from the vector is a chimera of tRNA and the RNA of interest.

Both boxes A and B of cand45 are well conserved, but by the same token can be considered ‘average’. See FIG. 11A. The strength of the cand45 expression system therefore is unlikely explained by boxes A and B alone, but by the overall sequence and configuration of cand45. The scope of the cand45 expression system of the present invention therefore preferably comprise a sequence either identical or substantially similar to that of the mature cand45 tRNA which serves to drive the expression of a downstream functional non-coding RNA of choice (small RNA, small hairpin RNA, microRNA, microRNA-like hairpin, ribozyme, antisense, structural RNA etc) and that is terminated by a Pol III terminator of choice.

In general, though not essential, upstream sequences have been shown to enhance tRNA expression. In some embodiments, ˜370 bp upstream of the mature cand45 tRNA is included in the cand45 derived expression vector of the present invention. However, a longer or shorter sequence may also be used in the present invention, as long as the sequence is derived from the cand45 gene, or is a homologous thereof, and drive the expression of the RNA at a proper level.

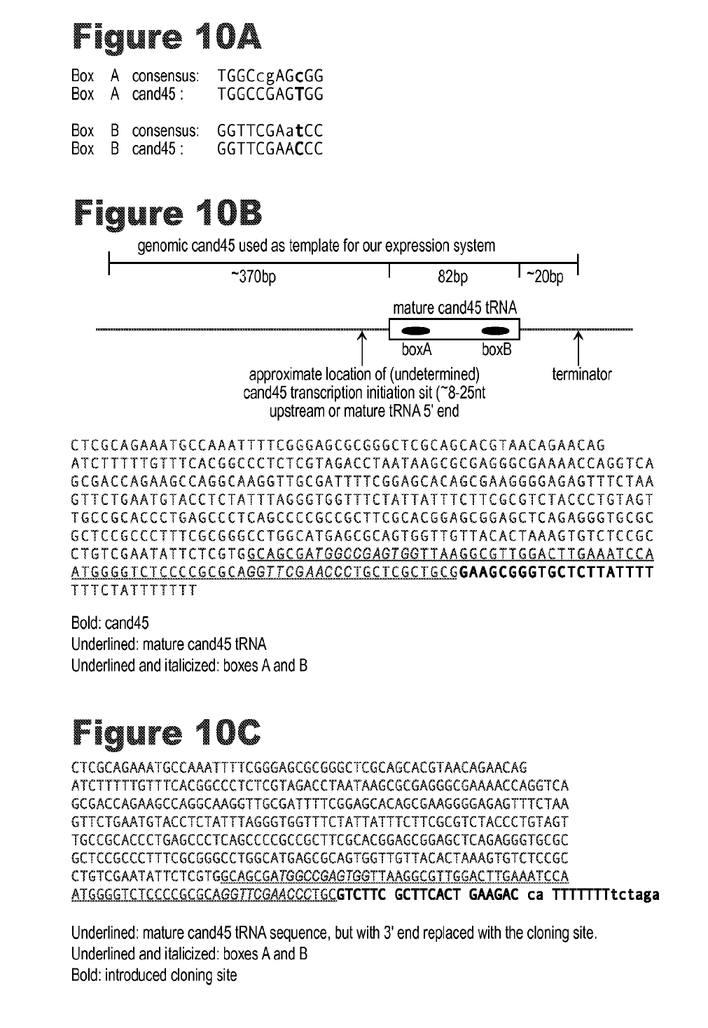

In some embodiments, the expression vector comprises 1-373 of the nucleotide sequence depicted in FIG. 11B, or a fragment of the 1-373 of the nucleotide sequence depicted in FIG. 11B. In some embodiments, the expression vector comprises sequence with about 60%, 65%, 70%, 75%, 80%, 85%, 90%, 95% or 100% identical to 1-373 of the nucleotide sequence depicted in FIG. 11B. When the sequence is identical to a fragment of the 1-373 of the nucleotide sequence depicted in FIG. 11B, the fragment is about 50, 100, 150, 200, 250, 300, or 350 base pairs long. Thus, the promoter sequence of the expression vector can be of different length and have a sequence different from the cand45 sequence shown in FIG. 10, as long as it can effectively drive the expression of the RNA to be expressed.

### V. Host Cells and Transgenic Animals

The present invention also provides host cells derived from a cell transfected with the expression vector described herein.

“Transfection” or “transformation” refers to a process by which agents are introduced into a cell. The list of agents that can be transfected is large and includes, but is not limited to, siRNA, sense and/or anti-sense sequences, DNA encoding one or more genes and organized into an expression plasmid, proteins, protein fragments, and more. There are multiple methods for transfecting agents into a cell including, but not limited to, electroporation, calcium phosphate-based transfections, DEAE-dextran-based transfections, lipid-based transfections, molecular conjugate-based transfections (e.g., polylysine-DNA conjugates), microinjection, viral gene transfer methods, and other methods known in the art.

Transfection can be either stable or transient. “Stable transfection” or “stably transfected” refers to the introduction and integration of foreign nucleic acid into the genome of the transfected cell. “Stable transfectant” refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). “Transient transfection” or “transiently transfected” refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. “Transient transfectant” refers to a cell that has taken up foreign nucleic acid but has not integrated this nucleic acid.

The host cells include, but are not limited to, bacteria, yeast cells, plant cells, insect cells, and mammalian (including human) cells. The host cell can be a differentiated cell, a transformed cell, or a stem cell.

The present invention also provides an animal comprising the host cells described herein. The host animal can be an insect, a fish, a bird or a mammal, e.g., a mouse, a rat, a rabbit, a dog, a monkey or a human being. The host animal can be generated using methods known in the art.

### VI. Applications

The systems and methods of the present invention find use in a variety of applications. In some embodiments, they are used to provide therapeutics for treating diseases. In some embodiments, they are used in a cell-based assay in drug target discovery and validation. In some embodiments, they are used for generating animal models for disease and treatment.

Potential therapeutic applications include neurodegenerative diseases such as Huntington's disease. After introduction of a vector directing the expression of the first strand of the invention with complementarity to Huntington mRNA into cells of the central nervous system expressing this gene, the complementary second strand may be applied from time to time to induce silencing of the disease-associated Huntington mRNA. This is expected to ameliorate the disease phenotype.

Another therapeutic application may be for treating cardiovascular disease. After introduction of a vector directing the expression of the first strand of the invention with complementarity to cholesterol-associated genes such as Apolipoprotein B or PCSK9 into hepatocytes expressing these genes, the complementary second strand may be applied from time to time to induce silencing of Apolipoprotein B or PCSK9 which is expected to lower levels of atherogenic lipids.

In another aspect, the present invention provide compositions and methods for treating diseases by modulating the level of tsRNA. In some embodiments, the expression level of a tsRNA in a disease condition is compared with the expression level of the same tsRNA in a normal condition. If there is a correlation between the altered expression level of the tsRNA and the disease condition, the tsRNA is modulated using the compositions and methods provided here to treat the diseases. One aspect of the invention relates to a method of treating a subject at risk for or afflicted with unwanted cell proliferation, e.g., malignant or nonmalignant cell proliferation, in which tsRNAs are known to be dysregulated. By “dysregulated” herein is meant the expression of a tsRNA, or a plurality of tsRNA, is altered (i.e., increased or decreased, in a disease condition) in comparison to the expression of such tsRNA in a normal condition. The method comprises providing a composition of the invention to modulate one or more tsRNA; and administering a therapeutically effective dose of the composition to a subject, preferably a human subject.

In some embodiments, the treatment comprises increasing or decreasing the activity of natural tsRNAs on a global level. In some embodiment, the treatment comprise inhibiting factors such as RNA polymerase III (Pol III), Pol III transcription factors such as Brfl, or RNaseZ to decrease the output of natural tsRNAs in general. In some embodiments, wherein an increase in tsRNAs was desired, global tsRNA levels are increased by augmenting the activity of Pol III or through the addition of cellular growth factors known in the art.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### EXAMPLES

### Example 1

### Tissue Culture

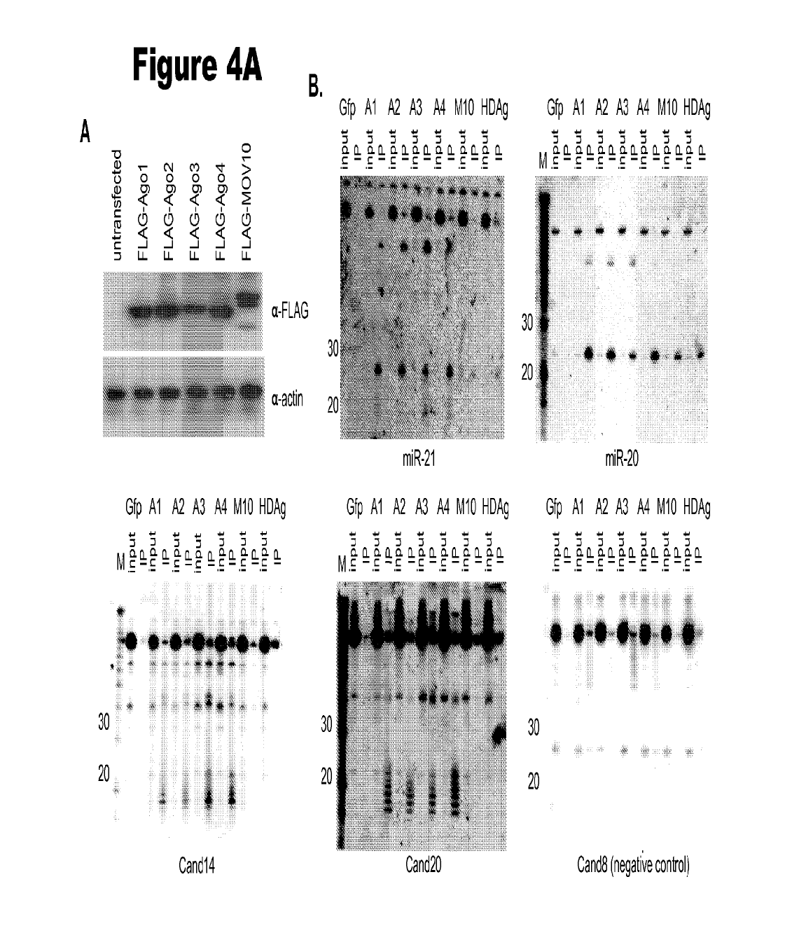

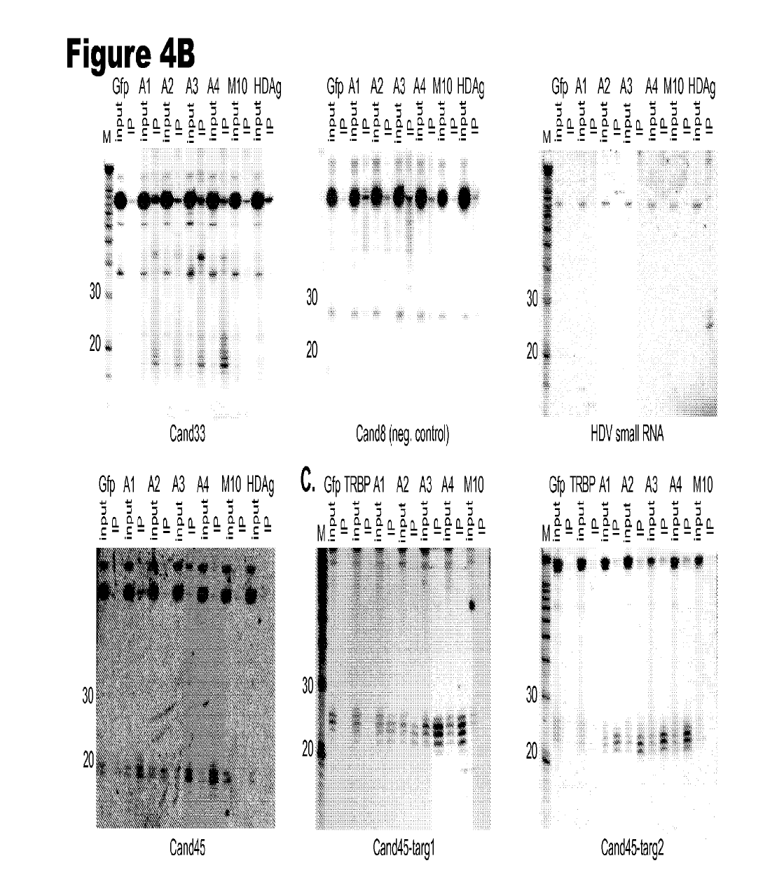

Human 293 and HCT116 cells (wild-type and derived Dicer helicase mutant cells were a kind gift from B. Vogelstein, John Hopkins, MD) were maintained in standard 10% FCS DMEM medium. For the RNA immunoprecipitation experiment in FIG. 4, the 293-derived FLAG-HDAg expressing cell line and the induction of HDV replication by plasmid transfection have been described before (Haussecker et al. 2008). Cells were transfected with Lipofectamine 2000 (L2K, Invitrogen) for plasmid DNA and RNAiMax (Invitrogen) for siRNA according to the manufacturer's instructions. For the serum starvation experiments, HCT116 cells were cultured at the indicated serum concentrations by daily media change and split such that they were growing at comparable cell densities. For the Brfl over-expression experiments, 293 cells were transfected with a Brfl over-expression plasmid on days 0 and 2 with 2 μg of plasmid DNA per 6 cm dish. For testing steady-state small RNA levels as a function of serum concentrations/Brfl over-expression, RNA was isolated for Northern blot analysis on day 5; for testing small RNA trans-silencing capacity under these conditions, luciferase reporter constructs were introduced on day 4 of serum starvation/Brfl over-expression and dual luciferase assays performed the next day.

### Plasmids

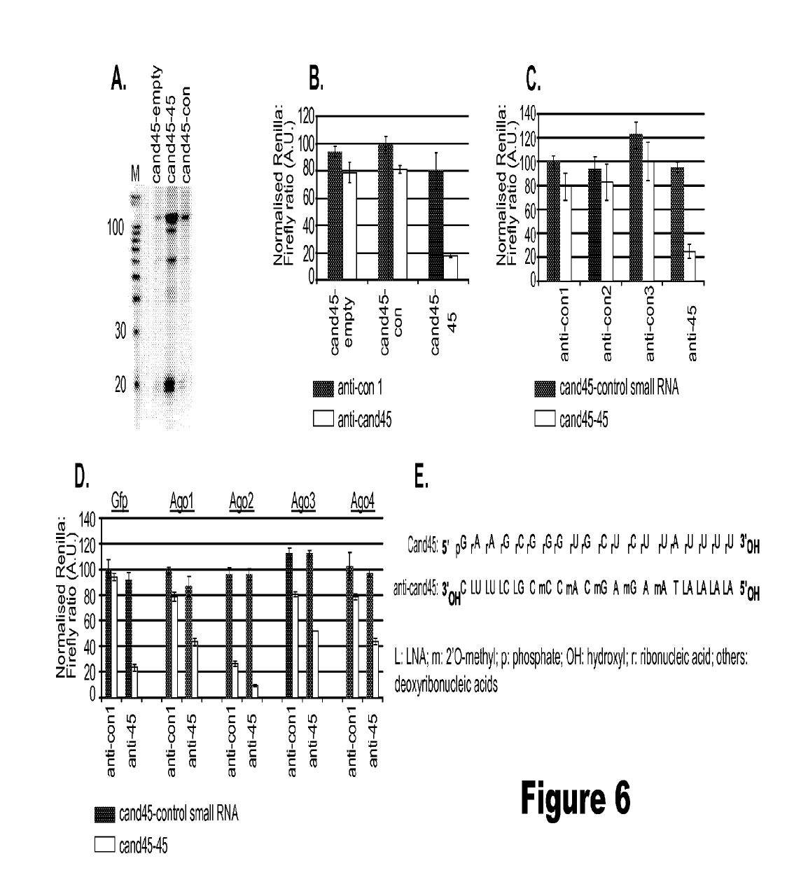

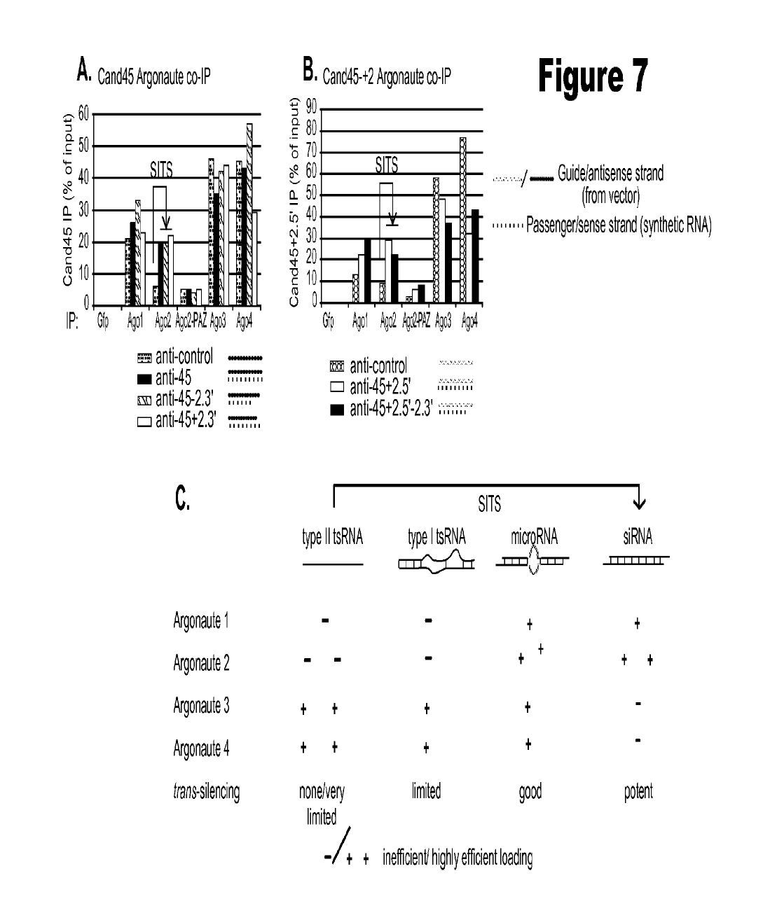

For RNA analyses, 2 μg expression plasmids were used per 6-well of 293 cells and RNA analyzed 48 hours after transfection. FLAG-Argonautes 1 and 2 and FLAG-EGFP are as described in Meister et al., 2004. To obtain similar expression levels for FLAG-Argonautes 1-4, the FLAG-Argonautes 3 and 4 described in Meister et al., 2004 were codon optimized by GENEART AG (Regensburg, Germany). Codon optimization did not change amino acids, only expression levels; detailed sequence available upon request. The FLAG-Ago2 PAZ deletion mutant in FIG. 7 was generated according to Gu et al., 2009 (manuscript submitted). FLAG-Mov10 is as described in Meister et al., 2005, FLAG-TRBP as described in Kok and Jin, 2007; pCMV-Dicer was a gift by Ian G. Macara (University of Virginia). A cand45 cloning vector (‘cand45-empty’) genomic sequence was amplified with primers hsCand45gen-F cttaaAAGCTTaagcttCTCTCGCAGAAATGCCAAAT and hsCand45gen-R cttaatctagaAAAAAAAtgGTCTTCAGTGAAGCGAAGACgcaggg TTCGAACCTGCGCGGGGAGAC and cloned into the HindIII-XbaI sites of pCRII-TOPO (Invitrogen). For cand45-45, cand45-targ.1 (referred to as ‘cand45-con’ in FIG. 6C), cand45-targ.2, cand45-+2.45, cand45-+4.45, and cand45-2.45 overexpression, the cloning vector was digested with BbsI, de-phosphorylated, and the following phosphorylated and annealed oligos inserted: ccctGCTCGCTGCGGAAGCGGGTGCTCTTA and aaaaTAAGAGCACCCGCTTCCGCAGCGAGC (cand45-45); ccctGCTCGCTGCGttcagcccgtcctctaggc and AAAAgcctagaggacgggctgaaCGCAGCGAGC (cand45-targ.1); ccctGCTCGCTGCGctcctcgagcgtcagacgc and AAAAgcgtctgacgctcgaggagCGCAGCGAGC (cand45-targ.2); ccctGCTCGCTGCGGAGAAGCGGGTGCTCTTA and aaaaTAAGAGCACCCGCTTCTCCGCAGCGAGC (cand45-+2.45); ccctGCTCGCTGCGGAGAGAAGCGGGTGCTCTTA and aaaaTAAGAGCACCCGCTTCTCTCCGCAGCGAGC (cand45-+4.45); ccctGCTCGCTGCGAGCGGGTGCTCTTA and aaaaTAAGAGCACCCGCTCGCAGCGAGC (cand45-2.45). In the co-transfection studies in FIG. 4C, 2 μg of cand45 and 2 μg of FLAG-protein expression vectors were co-transfected into 6 cm dishes of 293 cells. The Brfl over-expression vector was created by cloning the Brfl ORF from pTRE2-Brfl (Marshall et al. 2008) into pcDNA3 just downstream of an N-terminal FLAG epitope (Cao et al. 2009), while in the negative control vector the Brfl ORF had been replaced with the truncated ORF of a replication-deficient early nonsense mutant version of HDAg (Haussecker et al. 2008).

### Northern Blot

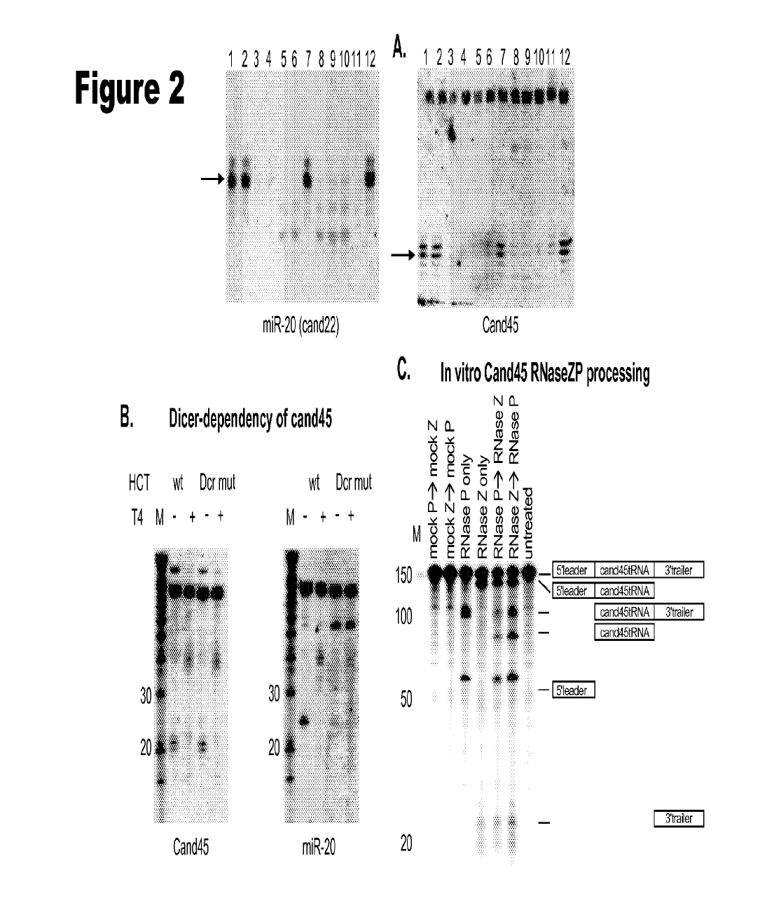

For small RNA Northern blotting, RNA was separated by 20% urea-polyacrylamide gel electrophoresis, transferred onto Hybond-N (Amersham) nitrocellulose by semi-dry transfer, and hybridised to T4 PNK end-labelled oligonucleotide probes overnight at 32° C. with PerfectHyb Plus (Sigma). Blots were washed 3 times with 6×SSC, 0.2% SDS (32° C., 34° C., 36° C.) and then once with 0.5×SSC, 0.1% SDS (42° C.) for 10 minutes each. Images were obtained by phosphorimager. Ambion's ‘Decade’ was used as a size marker. Northern probe oligos: gCACATGGTTAGATCAAGC (cand1); gAAAACCCACAATCCCTGGCTG (cand2); gAAAACCCACAATCCCTGGCTTA (cand3); gTCAATTAGTTGTAAACACCACTG (cand4); gTTCTAGGATAGGCCCAGGGGC (cand5); gCCAACTGAGCTAACCGGCC (cand6); gAACCCCACCAACATAGGGCTTCG (cand7); GGGCAGGCGAGAATTCTACCAC (cand8); GGATAACCACTACACTATGGAA (cand9); gTGGCGCCCGAACAGGGACA (cand10); GGCACCCCAGATGGGACACGA (cand11); gAAACGAGGTAACTCCGGA (cand12); GTGCCCGAGTGTGGTGGAGAATG (cand13); GAGTAGTGGTGCGTTGGCCGG (cand14); gTGGCGACCACGAAGGGACG (cand15); GAATTCTACCACTGAACCACAAT (cand16); GGCGACCACGAAGGGACACGA (cand17); GTTGTAAACACCACTGCACT (cand18); GGGCTTCAAAAAATTTGCTTGA (cand19); GGAGGGGGCACCCGGATTTGA (cand20); GGCGACCACGAAGGGACCCGA (cand21); gACTACCTGCACTATAAGCAC (cand22, aka miR-20); GGTGCGTTGGCCGGGAAACGA (cand23); gAAACAGCAAGCTAGTCAAGC (cand24); gCTTAGACCGCTCGGCCATCCTT (cand25); GACCGCTCGGCCACGCTACCCTC (cand26); gTGGCGAGCCAGCCAGGAG (cand27); gCCTTAGACCGCTCGGCCATCCT (cand28); GTCCTTGGTGCCCGAGGTGTCTA (cand29); GTGATATCCACTACACTACGGA (cand30); gCACCACTATACCACCAACGC (cand31); gCTCGCCAGGGCAAGGCTTACAA (cand32); GGTGCATGGGCCGGGAAACG (cand33); gACCACTGAACCACCAATGC (cand34); GGTTCCTGACCGGGAATCGAAC (cand35); GGTGCCGAAACCCGGGAACGA (cand36); gAACCCCACCAACATAGGGCTT (cand37); GTCCTTGGTGCCCGAGTGACCT (cand38); GACACCGTCCTTGGTGCCGCGT (cand39); GGACACCGTCCTTGGTGCCCAG (cand40); GCCCGAGGTGGTATGGCCGTAG (cand41); gTCTACCACTGAACCACCCATG (cand42); gACCACTGAACCACCCATGC (cand43); GAGAACCGTCCTTGGTGCCCGA (cand44); gAAAATAAGAGCACCCGCTTC (cand45); gCGAGGTAACTCCGGAGC (cand193); GAGGCACCTGCCAGGTGAC (cand401); gCTGAGCACAGGACTTCCTT (cand500); GAGCTTGGACGCTCGGTTGA (cand520); gTCGCCCTCTCAACCCAGCTTTT (sh320); gATCGGGAGGGGACTGAGCCTGA (sh484); gtcctggaaaccaggagtgc (MALAT mascRNA); AACTATACAACCTACTACCTCA (let-7a); acaaaccattatgtgctgcta (miR-15a); Gctatctgcactagatgcacct (miR-18a); Gtcaacatcagtctgataagc (miR-21); Gtcatagccctgtacaatgctg (miR-103); Gctacctgcactgtaagcacttt (miR-106a); Gcagctgcttttgggattccgtt (miR-191); gccatgctaatcttctctgtatc (U6 snRNA); AGAACTGGACAAAGTTTTCATCAC (sno38b); gcctagaggacgggctgaa (cand45-targ1); gcgtctgacgctcgaggag (cand45-targ2); ggcggcagtcctcagtactctta (HDV small RNA). For the Northern blot screen for the initial discovery of novel small silencing RNAs and the Dicer helicase mutant analysis in FIG. 2B, 4 μg mirVana (Ambion) low-molecular weight RNA was sequentially treated with TAP and/or T4 RNA ligase as described in ‘Analysis of small RNA 3’ and 5′ ends).

### Analysis of Small RNA 3′ and 5′ Ends

Enzyme treatments were performed by denaturing 4 μg mirVana (Ambion) RNA per sample at 65° C. for 5 minutes, chilled on ice for 2 minutes, followed by the addition of enzyme buffer, rRNasin (Promega; except for Terminator Exonuclease treatments) and finally enzyme. 15 μl reactions were incubated with the indicated enzymes at 37° C. (Terminator Exonuclease: 30° C.) for 60 minutes, acid phenol/chloroform extracted, ethanol precipitated and re-suspended for the second round of enzyme treatments which was again followed by acid phenol/chloroform extraction, ethanol precipitation and re-suspension in PAGE loading buffer for Northern blot. Buffer indicates that no enzyme was added. Amounts of enzymes used: 15 u of T4 PNK, 3′ phophatase +/−(NEB MO201/m0236); 8 units of Tobacco Acid Pyrophosphatase (Epicentre Biotechnologies); 3 units of Terminator Exonuclease (Epicentre Biotechnologies); 4 u polyA polymerase (PAP; Ambion); 15u T4 RNA ligase (NEB); for 3′ adapter ligation with T4 RNA ligase, a non-commercial buffer without ATP was made up and 1 μg of the following activated 3′ adapter added: 5′AppCTGTAGGCACCATCAAT-NH2 3′ (NEB 1315).

### RNA Immunoprecipitation

To test for the distribution of tsRNAs, cell lysate corresponding to a 6 cm dish of confluent 293 cells were used for each immunoprecipitation. Lysates were prepared by washing cells 48 hours after FLAG-protein transfection twice with ice-cold PBS and then lysing them with 0.6 ml/10 cm dish M-PER lysis buffer (Pierce) containing protease inhibitor cocktail (-EDTA, Roche). Lysates were diluted by the addition of 3× volumes IP buffer (20 mM Tris-HCl (pH8.0), 50 mM KCl, 0.2 mM EDTA, 10% glycerol). 1/10 of the volume lysate per immunoprecipitation was removed and RNA isolated with Trizol (Invitrogen) for the input RNA control. Per sample, 20 ul anti-Flag M2 agarose beads (Sigma A2220) was added to the lysate for incubation with rotation overnight at 4° C. The next day, immunoprecipitates were washed extensively with IP buffer and RNA isolated by the addition of Trizol to the beads. To delineate the mechanism of sense-induced trans-silencing, HCT116 cells were co-transfected in 6 cm dishes with 2 μg of FLAG-bait, 2 μg of the cand45 over-expression vector and 0.1 μg of the luciferase reporter, two hours after which 100 nM of the sense oligonucleotide was added. Cells were split the next day into one 6 cm dish and 3 24-wells each, with lysates harvested and processed for RNA immunoprecipitation (6 cm dish) or luciferase assay (24-wells) 48 hours after the first transfection.

### RNaseZP Processing Assay

RNaseZP processing assays were as described (Wilusz et al., 2008) Purified HeLa RNaseP and recombinant His-tagged tRNaseZL (delta30) were generously provided by Sidney Altman (Yale University, CT) and Masayuki Nashimoto (Niigata University, Japan), respectively. After the first enzyme treatments, RNA was acid phenol/chloroform extracted and ethanol immunoprecipitated, and re-suspended for performing the second enzyme reaction. Mock treatment was buffer without enzyme addition. An internally alpha-P32 UTP-labelled, gel-purified cand45 precursor tRNA substrate was prepared by SP6 in vitro transcription of XbaI-linearized plasmid SP6-cand45. SP6-cand45 was obtained by cloning into the HindIII-XbaI sites of pCRII-TOPO (Invitrogen) a genomic cand45 tRNA PCR fragment using primers SP6-cand45-F (cttaaAAGCTTACTAAAGTGTCTCCGCCTG) and SP6-cand45-R (ttaatctagaAAATAAGAGCACCCGCTTCCGCAGCGAGCAGGGTTCGAACCTGCGCGGGG; starts 34 nucleotides upstream of the predicted RNaseP cleavage site and ending with the RNA polymerase termination oligo-dT stretch).

### Nuclear-Cytoplasmic RNA Fractionation

Nuclear-cytoplasmic fractionation was performed as described (Haussecker et al. 2008). 2 μg of each fraction was used for Northern blot analysis.

### Dual Luciferase Assay

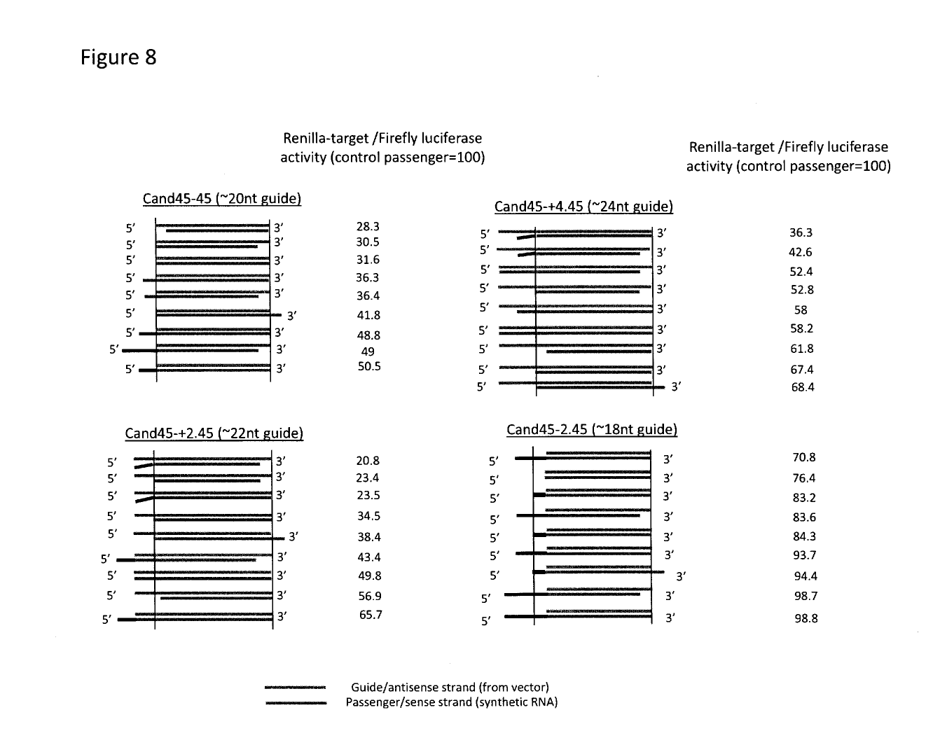

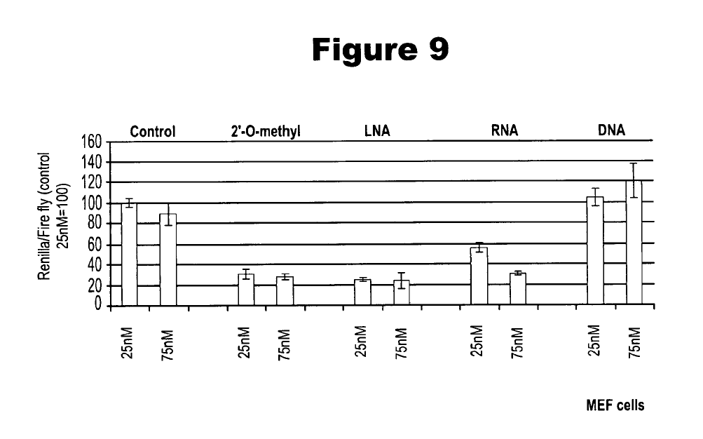

The Dual-Luciferase® Reporter (DLR™) Assay (Promega) was performed according to the manufacturer's instructions, with the following modifications. Cand14 and Cand45 reporter vectors were derived by inserting the following phosphorylated and annealed oligos into the XhoI-SpeI sites of Renilla luciferase in psi-check2 (Promega): TCGAcgagtagtggtgcgttggccgggaaAAAAAcgagtagtggtgcgttggccgggaa and CTAGttcccggccaacgcaccactactcgTTTTTttcccggccaacgcaccactactcg (psi-cand14); tcgaAAAATAAGAGCACCCGCTTCaaaaAAAATAAGAGCACCCGCTTC and ctagGAAGCGGGTGCTCTTATTTTttttGAAGCGGGTGCTCTTATTTT (psi-cand45 wt/aka ‘psi-cand45 wt 2×’ in Supplementary FIG. 6); TCGAAAAATAAGAGCACCCGCTTC and CTAGGAAGCGGGTGCTCTTATTTT (psi-cand45 wt 1×); tcgaAAAAAAAATAAGAGCACCCGCTTCTCTCaaaaAAAAAAAATAAGAGCACCCGCTT CTCTC and ctagGAGAGAAGCGGGTGCTCTTATTTTTTTTttttGAGAGAAGCGGGTGCTCTTATTTTTT TT (psi-cand45 wt_extended; FIG. 7 and Supplementary FIG. 9); TCGAaactatacaacctactacctcAaaaaaaactatacaacctactacctcAaaaaaaaactatacaacctactacctcA; ctagTgaggtagtaggttgtatagttttttttTgaggtagtaggttgtatagtttttttTgaggtagtaggttgtatagtt (psi-let-7aPM); TCGAaTcTAtTGaaGGAactacctcAaaaaaaaaTcTAtTGaaGGAactacctcAaaaaaaaaTcTAtTGaaGG AactacctcA; ctagTgaggtagtTCCUCAaTAgAtattatTgaggtagtTCCttCAaTAgAttttattTgaggtagtTCCttCAaTAgA t (psi-let-7aMM); TCGActacctgcactataagcactttaaaaaaactacctgcactataagcactttaaaaaaactacctgcactataagcacttta; ctagtaaagtgcttatagtgcaggtagtttttttaaagtgcttatagtgcaggtagtttttttaaagtgcttatagtgcaggtag (psi-miR-20PM); TCGActTGctGCaGtaTAagcactttaaaaaaactTGctGCaGtaTAagcactttaaaaaaactTGctGCaGtaTAagc acttta; ctagtaaagtgaTAtaCtGCagCAagttttataaagtgctTAtaCtGCagCAagtttattaaagtgctTAtaCtGCagCAag (psi-miR-20 mM); tcgaTAGTTTTCACAATGATCTCGGTAGTTTTCACAATGATCTCGGTAGTTTTCACAAT GATCTCGGTAGTTTTCACAATGATCTCGG and ctagCCGAGATCATTGTGAAAACTACCGAGATCATTGTGAAAACTACCGAGATCATTG TGAAAACTACCGAGATCATTGTGAAAACTA (psi-bantam). Each transfection was performed in triplicate 24-wells by co-transfecting the following amounts of nucleic acids with L2K into 293 (cand14 assays) or HCT116 (cand45 assays) cells: 50 ng psi-check reporter plasmid, and either 225 ng+225 ng FLAG-protein expression+cand45-45/-targ.1/-targ.2 or 450 ng FLAG-protein expression vector. 100 nM antisense oligonucleotides were applied 2 hours after plasmid transfection, lysates harvested for luciferase assays 48 hours after the tranfections. With the exception of the experiment shown in Supplementary FIG. 7, the Renilla:Firefly luciferase ratios of the target vectors were further normalized to the Renilla:Firely luciferase ratio of a psi-check2-derived reporter in which the tsRNA target sequences were replaced by bantam microRNA target sequences (not expressed in human cells). Antisense oligonucleotides: 5′-+T+A+G+T+G G mU G mC G mU T mG+G+C+C+G G (anti-14); 5′-+A+A+A+A TmA AmGA mGCmA CmCC+G+C+T+TC-3′ (anti-cand45/anti-45 LNA/methyl); 5′-mAmAmA mAmUmA mAmGmA mGmCmA mCmCmC mGmCmU mUmC-3′ (anti-cand45-methyl); 5′-+A+A+A+A+U+A+A+G+A+G+C+A+C+C+C+G+C+U+U+C-3′ (anti-cand45-LNA); AATGGCCTCGAGCCTCCTCAATTCACAACCTG (anti-con1); mAmGmG mCmGmG mCmAmG mUmCmC mUmCmA mGmUmA mCmUmC mUmUmA (anti-con2); mUmAmA mGmAmG mUmAmC mUmGmA mGmGmA mCmUmG mCmCmG mCmCmU (anti-con3). Anti-cand45 oligonucleotides for testing structure-function of sense-induced trans-silencing (FIG. 8): 5′-mAmAmA mAmUmA mAmGmA mGmCmA mCmCmC mGmCmU-3′ (anti-cand45, 2 nt matching cand45 5′ end removed); 5′-mAmAmA mAmUmA mAmGmA mGmCmA mCmCmC mGmCmU mUmC mUmC-3′ (anti-cand45 with additional 2 nucleotides matching cand45-derivative cand45-+2.45); 5′-mAmAmA mAmUmAmAmGmAmGmCmAmCmCmCmGmCmUmUmCmUmCmUmC-3′ (anti-cand45 with additional 4 nucleotides matching cand45-derivative cand45-+4.45); 5′-mAmAmAmAmA mAmUmA mAmGmA mGmCmA mCmCmC mGmCmU mUmC-3′ (anti-cand45 with 2 additional nucleotides that would be able to match a cand45-derivative with extended oligo-U 3′ tail); 5′-mA mAmUmA mAmGmA mGmCmA mCmCmC mGmCmU mUmC-3′ (anti-cand45 resulting in 2 nucleotide 3′ overhang of cand45 with 4Us at 3′ end); 5′-mA mAmUmAmAmGmAmGmCmAmCmCmCmGmCmUmUmCmUmCmUmC-3′ (anti-cand45 with additional 4 nucleotides matching cand45-derivative cand45-+4.45 and resulting in 2 nucleotide 3′ overhang of cand45 with 4Us at 3′ end); 5′-mA mA mA mA mU mA mA mG mA mG mC mA mC mC mC mG mC mU mU mC mC mG-3′ (anti-cand45 with 3′ extension that would extend complementary towards cand45 2 nucleotides upstream of predicted RNaseZ cleavage site); 5′-mA mAmUmA mAmGmA mGmCmA mCmCmC mGmCmU mUmC mUmC-3′ (anti-cand45 with 3′ extension that would extend complementary towards cand45 2 nucleotides upstream of predicted RNaseZ cleavage site and resulting in 2 nucleotide 3′ overhang of cand45 with 4Us at 3′ end); mAmAmCmUmAmUmAmCmAmAmCmCmUmAmCmUmAmCmCmUmCmA (Let-7a antisense); miRIDIAN miR-20 Hairpin Inhibitor: Dharmacon Cat. No. 1H-300491-05: miRIDIAN let-7a Hairpin Inhibitor: Dharmacon Cat. No. 1H-300473-07; +: LNA; m: 2′-β-methyl; others: DNA bases.

### RNAi

293 (Brfl) and HCT116 (serum) cells were cultured for 4 days under the indicated conditions and then transfected with siRNAs targeting the endogenously expressed RALY gene using RNAiMax (Invitrogen). RNA was harvested the next day with Trizol (Invitrogen) and quantitative reverse transcription realtime PCR (qRT-PCR) performed according to Haussecker and Proudfoot, 2005; actin was used for normalization. Two nucleotide dTdT 3′ overhang siRNAs of the following sequences (sense/passenger) were obtained from Dharmacon/Thermo Fischer: GAUCAAGUCCAAUAUCGAUdtdt (si-1); GCGUGUCAAAACUAACGUAdtdt (si-2); AGACGACGGCGAUGAGGAAdtdt (si-3); RT-PCR primers: cttaatgtcacgcacgatttcc (actin RT); aaatctggcaccacaccttc (actin forward); agaggcgtacagggatagca (actin reverse); tcttcctcgctgtgtgtcag (RALY RT); ttctgtgcacaagggctatg (RALY forward); atggcagatgctgctctctt (RALY reverse).

### Western Blot

Western Blot was performed according to standard protocols. 2 μg of protein from 293 cells transfected with the indicated expression plasmids were run on 4-20% polyacrylamide gradient gels, blotted, and probed with the following antibodies: mAb FLAG M2 (Sigma, A8592); mAb anti-actin (Sigma, A5316).

### Example 2

### Small RNA Screen Uncovers 5′-phosphate, 3′-hydroxyl tRNA-Derived Small RNAs

In a previous study, we reported on the discovery of two HDV small RNAs, 20-25 nucleotides in length and with mRNA-like cap structures (Haussecker et al. 2008). While the antigenomic small RNA was only seen by 5′ phosphate-dependent semi-deep sequencing in an RNA preparation enriched for 5′ capped RNAs consistent with biochemical analyses, a corresponding small RNA of genomic polarity was found with and without prior 5′ cap enrichment. Based on the hypothesis that this might be a reflection of the function of this particular class of small RNAs, we set out to discover cellular counterparts of the HDV small RNAs through a Northern blot screen with probes targeting particularly those sequences that occurred in both the 5′ cap-enriched and non-enriched samples. Further selection criteria were a high sequencing frequency to facilitate their subsequent analysis, and not being annotated as obviously deriving from known and abundant non-coding RNAs. The selected candidates included a number of sequences without a perfect match to the genome (nuclear or mitochondrial), as has also been observed, but largely excluded from further analysis in previous small RNA sequencing studies (e.g. Azuma-Mukai et al. 2008).

To screen for novel small RNAs based on their end modifications, RNA from the human embryonic kidney cell line HEK 293 was treated with the decapping enzyme Tobacco Acid Phosphatase (TAP) and/or T4 RNA ligase. While TAP removes 5′ caps, T4 RNA ligase can circularize 5′ phosphorylated, 3′ hydroxylated RNAs or ligate RNAs that contain either of these end structures to each other in trans. RNAs that have participated in these reactions are marked by either a shift in their gel mobility or their disappearance. 44 of the 45 probes used in the screen, including those directed at sequences for which no perfect genomic match had been initially identified, readily detected RNAs with an apparent size of 70-150 nucleotides (data not shown) (We note that probes with random sequences do not recognize such RNAs under the experimental conditions applied (data not shown). Based on their size and apparent abundance, we expected that the detected RNAs might include non-coding RNAs such as members of the tRNA, snoRNA, and snRNA families. We speculate that the lack of perfect matches to the human genome for many of the sequences could be due to post-transcriptional modifications (well characterized for numerous non-coding RNAs) and consequent nucleotide changes as a result of mis-incorporations during the reverse transcription step of cDNA library preparation (Kawaji et al. 2008). A recent analysis of apparent RNA sequencing errors strongly supports this notion (Ebhardt et al. 2009).

None of the probes detected 20-25 nucleotide small RNAs that shifted upon TAP treatment, indicating a lack of predominantly capped small RNAs and that sequencing alone is not sufficient to conclusively deduce end modifications. Instead, we noticed a number of T4 RNA ligase-sensitive RNAs, 12 out of the 45 candidate sequences, that, strikingly, were in the 20-22 nucleotide size range, the typical length of small silencing RNAs. Many of the same probes also detected a number of larger and smaller RNAs around the 20-22 nt size range which, however, were largely insensitive to T4 RNA ligase treatment (for example cand14 and 33; data not shown) and varied in intensity from experiment to experiment, consistent with these being T4 RNA ligase-insensitive degradation products. The T4 RNA ligase-sensitive small RNA detected with the probe directed at candidate 45 (cand45) appeared to be distinct from the other T4 RNA ligase-sensitive small RNAs in that its intensity was comparable to that of its larger, ˜110 nucleotide counterpart (FIG. 2A). Further enzymatic analysis confirmed that these small RNAs, in notable contrast to the T4 RNA ligase-insensitive RNAs, were indeed 5′-phosphorylated and 3′-hydroxylated (see FIG. 2A for cand45 example). Accordingly, similar to a control 5′-phosphorylated, 3′-hydroxylated microRNA (miR-20/cand22), treatment of all five accordingly investigated candidates (cand14, 23, 33, 35, 45) with the Terminator nuclease (lane 4), an exonuclease that degrades (unstructured) 5′ phosphorylated RNAs, selectively removed the T4 RNA ligase-sensitive RNAs; 3′ adapter ligation with an activated 3′ adapter and in the absence of ATP led to the disappearance of the T4 RNA ligase-sensitive small RNAs (lane 8), as did treatment with polyA polymerase (lane 11), both of which are indicative of 3′ hydroxyl ends.

While these experiments were ongoing, a bioinformatic paper by Babiarz and colleagues reported on a population of tRNA-derived small RNAs in mouse embryonic stem cells that were Dicer-dependent, but Drosha-independent (Babiarz et al., 2008). Closer inspection of the T4 RNA ligase-sensitive sequences strongly suggested that most, if not all of them were indeed tRNA-derived. Firstly, 8 out of the 12 T4 RNA ligase-sensitive small RNA sequences contained a tRNA-like ‘CCA’ motif at their 3′ ends. Some of the reported Dicer-dependent tsRNAs had similarly been reported to be CCA-ylated at their 3′ ends (Babiarz et al., 2008). Secondly, when blasted manually, perfect genomic matches could now be identified for candidates 11, 15, and 35 and were found to be part of predicted tRNAs. Moreover, in the absence of a perfect match to the genome, candidates 14 and 23 had originally been tentatively annotated as derived from endogenous retroviral elements (ERV). This is reminiscent of the human tRNA-derived RNAs reported by Kawaji and colleagues, some of which appeared to be mis-annotated as endogenous retroviral elements due to the role that tRNAs play in the replicative priming of retroviruses and nucleotide mis-incorporations opposite of modified bases during the reverse transcription step of cDNA library preparation (Kawaji et al., 2008). The absence of RNAs detected with probes directed at the antisense strand of the T4 RNA ligase-sensitive small RNAs (data not shown) is consistent with the previous suggestion (Babiarz et al., 2008) that imperfectly base-paired tRNA stem structures rather than paired sense-antisense transcripts were the substrates for Dicer processing. We note that while Dicer-dependent tsRNAs had been identified in mice based on sequencing and bioinformatic prediction, their detection by alternative methods as well as further structural and functional analysis has been lacking. As we will illustrate, the ability to identify them based on their T4 RNA ligase- and Terminator-sensitivities in Northern blots facilitates such analyses.

Two Types of tRNA-Derived Small RNAs

The absence of RNAs detected with probes directed towards the antisense strand of the T4 RNA ligase-sensitive small RNAs (data not shown) is consistent with the previous suggestion (Babiarz et al. 2008) that imperfectly base-paired tRNA stem structures rather than paired sense-antisense transcripts were the substrates for Dicer processing. We note that while Dicer-dependent tsRNAs had been identified in mice based on sequencing and bioinformatic prediction, their direct detection by alternative methods as well as further structural and functional analysis had been lacking. Manual BLAST analysis of candidate 45 mapped its 5′ end to directly downstream of the discriminator base of a predicted serine-tRNA, and ending in a short stretch of uracils. This immediately suggested a Dicer-independent mode of biogenesis in which the 5′ and 3′ ends are determined by the tRNA processing enzyme RNaseZ, an endonuclease leaving a 3′-hydroxyl and 5′ phosphate at the cleavage site (Mayer et al. 2000), and transcription termination by RNA polymerase III, respectively. Accordingly, we failed to detect a decrease in cand45 abundance either when 293 cells were treated with Dicer siRNAs (although insufficient Dicer knockdown could not be ruled out; data not shown), or in a HCT116-derived human colorectal cancer cell line in which the Dicer helicase domain had been mutated leading to a decrease in most, albeit not all microRNAs (FIG. 2B; Cummins et al. 2006). In contrast, treatment of an in vitro transcribed cand45 tRNA precursor with recombinant RNaseZ yielded the predicted 3′ trailer small RNA (FIG. 2C). Interestingly, at least in this assay, the efficiency of 3′ processing was independent of whether the precursor tRNA had been pre-treated with RNaseP or not. RNaseP is the enzyme that cleaves off the 5′ leaders of precursor tRNAs and may be required for the functioning of RNaseZ for at least some tRNAs (Dubrovsky et al. 2004; Nashimoto et al. 1999). We will refer to this type of tsRNA as type II tsRNA, as opposed to the Dicer-dependent type I tsRNAs.

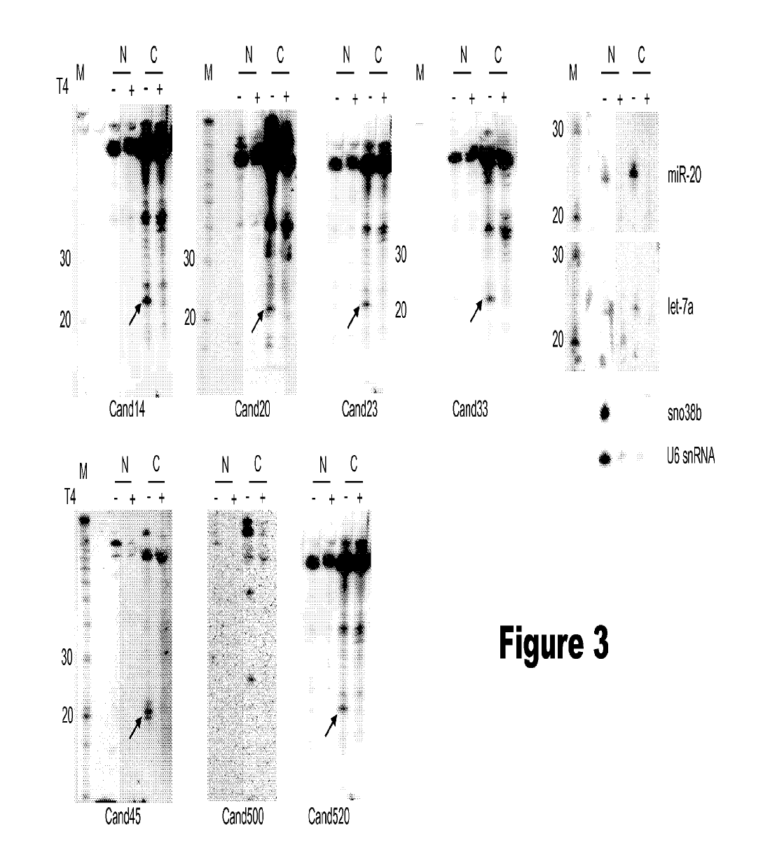

Removal of the 3′ trailers of nuclear encoded pre-tRNAs by RNaseZ is thought to take place in the nucleus (Lund and Dahlberg 1998). It is therefore notable that following nuclear-cytoplasmic fractionation essentially all detectable cand45 tsRNA was recovered in the cytoplasmic fraction (FIG. 3). We confirmed our fractionation results by examining the distribution of known RNA markers. In particular, both positive controls for nuclear RNAs tested, snoRNA 38b and U6 snRNA, were almost entirely restricted to the nuclear fraction. This suggests that cand45 is either rapidly exported following RNaseZ cleavage or that a population of cytosolic RNaseZ that has been described to function as the effector endonuclease in a new type of gene silencing guided by 5′ half tRNAs (Elbarbary et al. 2009), may be responsible for RNaseZ-dependent cand45 biogenesis in the cytoplasm. Like cand45, the four type I tsRNAs that we investigated (cand14, 20, 23, 33), which could be identified by size and T4 RNA ligase-sensitivity were each almost exclusively detected in the cytoplasmic fraction and is consistent with cleavage by cytoplasmic Dicer. Of note, while tsRNAs were absent from the nuclear fraction, a portion of microRNAs was always detected in the nucleus (miR-20 and let-7a in FIG. 3), albeit at generally lesser intensity than their cytoplasmic counterparts. This is consistent with other observations that microRNAs can be readily detected in the nuclei of mammalian cells (Hwang et al. 2007). More importantly, however, the distinct fractionation

tRNA-Derived Small RNAs Have Relative Preference for Argonaute 3 and 4 Association

The interpretation of tsRNAs forming a distinct population of small RNAs was also consistent with our analysis of the interactions of tsRNAs and microRNAs with the small RNA effector proteins Argonautes 1-4 and the microRNA factor Mov10. To facilitate this analysis, FLAG-tagged versions of the various proteins were expressed with comparable efficiencies in 293 cells (FIG. 4A) so that associated small RNAs could be immunoprecipitated with the same monoclonal FLAG antibody across all cell lysates. The use of FLAG-tagged Argonautes in elucidating bona fide Argonaute function and small RNA association has been well validated in previous studies. Accordingly, such epitope-tagged Argonautes co-fractionate with their endogenously expressed counterparts (Hock et al. 2007), no obvious changes in the small RNA profiles were observed following transient overexpression of a FLAG-tagged Argonaute 2 (Zhang et al. 2009), and no gross differences were noted in small RNA immunoprecipitations with antibodies against endogenous Argonautes when compared with earlier studies immunoprecipitating FLAG-tagged Argonautes (Azuma-Mukai et al. 2008; Ender et al. 2008). On the other hand, overexpression experiments with similar Argonaute constructs have been shown to result in competition with endogenously expressed Argonaute function (Diederichs et al. 2008). A conservative interpretation of such Argonaute co-IP experiments would be therefore that they illustrate the relative abilities of Argonautes to load various small RNAs under conditions when they are not limiting. FLAG-Gfp and cand8 served as negative controls for non-specific FLAG-protein interactions and for non-T4 RNA ligase-sensitive small RNAs (i.e. small RNAs presumably unrelated to RNAi), respectively.

All type I tsRNAs tested (cand14, 20, 23, 33) were readily immunoprecipitated with FLAG-Argonautes 1-4, but could not be detected following FLAG-Mov10 immunoprecipitation (FIG. 4B). This is in contrast to the investigated microRNAs miR-20, miR-21, and let-7a which co-immunoprecipitated also with Mov10 (FIG. 4B), albeit to a lesser extent than with the Argonautes. Interestingly, only in the case of type I tsRNAs, there were in addition to the 21-22 nt species identified in the original screen smaller, T4 RNA ligase-sensitive (data not shown) 18-20 nt RNAs detected with the same oligonucleotide probes that were even more efficiently enriched in the Argonaute immunoprecipitates. The type II tsRNA cand45 similarly co-immunoprecipitated with all Argonautes and could not be detected following Mov10 immunoprecipitation. In this case, however, no <20 nt species was enriched in the immunoprecipitates. Moreover, unlike microRNAs which appeared to co-immunoprecipitate equally well with all the Argonautes, cand45 associated more efficiently with Argonaute 3 and 4 than with Argonautes 1 and 2 (FIG. 4B). The same Argonaute 3-4 over Argonaute 1-2 preference was observed when the cand45 sequence downstream of the predicted RNaseZ site was replaced with two arbitrary sequences and expressed in 293 cells (Cand45-targ 1 and Cand45-targ2; FIG. 4C). This suggests that the particular pathway/biogenesis, not the sequence per se determines the Argonaute association pattern. We also tested tsRNA association with a FLAG-tagged version of the Hepatitis Delta Virus Antigen (HDAg) which we had reported to be associated with both Mov10 and HDV small RNAs (Haussecker et al. 2008). Only very little tsRNAs were immunoprecipitated with FLAG-HDAg (<5% of Ago-4 immunoprecipitation) suggesting that the tsRNA-associated Argonaute pool was also distinct from the HDV-related small RNA pathway. The FLAG-HDAg co-immunoprecipitation of microRNAs 20 and 21 could reflect its association with Mov10. In summary, in addition to the nuclear-cytoplasmic fractionation patterns, tsRNAs are further differentiated from microRNAs by their apparent lack of Mov10 association. Moreover, while our overexpression studies may not quantitatively recapitulate the normal distribution patterns of tsRNAs between the Argonautes, they at the very least demonstrate a relative propensity of the type II tsRNA pathway for Argonaute 3 and 4 utilization.

Abundance and Size of Type II tsRNA-Like Small RNAs are Sensitive to Argonaute Dosage

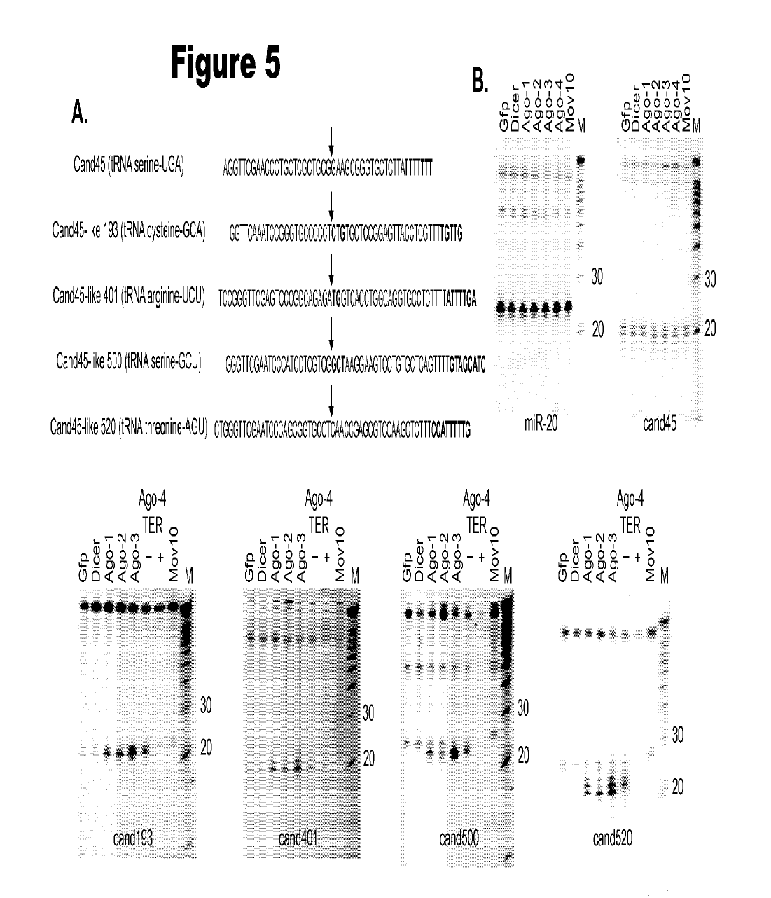

To search for additional cand45-like type II tsRNAs that may have been generated via RNaseZ cleavage and RNA polymerase III termination, we examined four candidates in our small RNA database (cand193, 401, 500, and 520) that met the criteria of mapping to the 3′ ends of predicted tRNAs and ending in a stretch of uracils (FIG. 5A). However, unlike cand45, none of the 5′ ends for these candidates coincided precisely with the predicted major RNaseZ cleavage sites, with those of cand193, 401, and 500 mapping slightly upstream, and that of cand520 downstream of the RNaseZ site. Northern blot analysis confirmed the existence of corresponding small RNAs (FIG. 5B). Cand500 and 520 were identified as Terminator-sensitive and therefore 5′ phosphorylated small RNAs of 24-28 nucleotides, while cand193 and 401 yielded 5′ phosphorylated 20-21 nucleotide RNAs. Like type I and II tsRNAs, cand45-like small RNAs were predominantly localized to the cytoplasm (see FIG. 3 for cand500 and cand520 examples). As RNAi knockdown of RNAi components to levels that affected the steady-state abundance of microRNAs proved challenging in 293 cells (data not shown), we sought to investigate the relationship between RNAi and tsRNA pathways by testing the effect of overexpressing Dicer, Ago1-4, and Mov10 on small RNA abundance. Interestingly, all four cand45-like small RNAs were affected when overexpressing Argonautes 1-4 (FIG. 5B). Accordingly, Argonaute overexpression robustly increased the abundances of the 20-21 nucleotide long cand193 and 401 RNAs, by ˜20-fold in the case of Argonaute 3 overexpression which showed the strongest effect. In contrast, while the abundances of the 24-28 nt cand500 and 520 RNAs were essentially unchanged, Argonaute overexpression triggered the appearance of 21-23 nucleotide RNAs that were readily detected by the same probes and that were of higher abundance than the uninduced longer counterparts. One possible explanation may be that the proximity of Argonautes with tRNA 3′ processing allowed Argonautes, acting as molecular rulers (Wang et al. 2008), to selectively capture and stabilize the tsRNAs. This is consistent with the observation that although both the induced shorter and non-induced longer cand45-like small RNAs co-immunoprecipitated with Argonautes (but not Mov10), the process was more efficient for the induced species (data not shown). Alternatively, the induced species may be the result of a catalytic activity of Argonautes upstream of target cleavage in the RNAi pathway, as has been suggested for Argonaute 2 (O'Carroll et al. 2007; Diederichs and Haber 2007). Importantly, the abundance of cand45 itself was not affected by Argonaute overexpressions, as was the case for microRNAs 20 and 21 and the bioinformatically predicted endo-shRNAs 320 and 484 (Babiarz et al. 2008), arguing that these are already efficiently incorporated into Argonautes at physiological expression levels. It will be interesting to test whether the differences in Argonaute responsiveness is determined by whether or not the 5′ end of the tRNA trailer-derived small RNAs precisely coincides with the predicted major RNaseZ cleavage site.

Differential Trans-Silencing Capacity of Type I and II tsRNAs

The post-transcriptional trans-silencing capacity of tsRNAs was tested using standard reporter assays (FIG. 6). Specifically, target sites complementary to the type I and II tsRNAs cand14 and 45, respectively, were inserted into the 3′ UTR of a Renilla luciferase reporter gene. These constructs were then transfected into tissue culture cells expressing the tsRNAs. Renilla luciferase activity was normalized for the luminescence of a firefly luciferase reporter gene on the same plasmid as well as for the Renilla-Firefly ratio of a second plasmid that lacked a tsRNA target site at the corresponding position of the Renilla 3′ UTR. Modest cand14 trans-silencing capacity was deduced based on the ˜30-40% increase in Renilla activity when cells were treated with a cand14 antisense oligonucleotide compared to all four negative control (antisense) oligonucleotides tested (data not shown). It is possible that the trans-silencing capacity of cand14 is inherently modest, perhaps reflecting preferential Ago3-4 over Ago1-2 interaction (FIG. 4B). It is, however, also possible that the extent of the silencing was limited either due to the specific context of the reporter or as the target site might not have been entirely complementary to cand14 based on the absence of a perfect match in the genome for cand14. Similarly, since simple antisense-inhibition of small RNAs, however, may not necessarily be the most efficient way of antagonizing their activities (Vermeulen et al. 2007), we cannot be certain that the antisense-dependent up-regulation reflects the full degree of cand14-mediated trans-silencing. In summary, cand14 exhibits canonical microRNA-/siRNA-like trans-silencing capacity.

In contrast to the type I tsRNA cand14, cand45 did not exhibit apparent trans-silencing activity (FIG. 6B). Even when Argonautes were overexpressed (FIG. 6D), there was no strong cand45 antisense-reversible trans-silencing, although Argonaute 2 overexpression caused a small 20-30% reduction in reporter gene activity in some datasets, which was reversed by an oligonucleotide antisense to cand45. We reasoned that by overexpressing cand45 we would be able to achieve cand45-dependent gene silencing. For this, a cloned version of the cand45 tRNA was transfected into HCT116 cells and

cand45 overexpression confirmed by Northern blot (FIG. 6A). Nevertheless, the co-transfected Renilla luciferase reporter was still not silenced (FIG. 6B). Unexpectedly, when a cand45 antisense oligonucleotide was added (100 nM), originally intended to relieve any cand45-mediated trans-silencing, there was a robust induction of cand45-Renilla luciferase silencing that was further responsive to the number of target sites in the reporter (an over 80% decrease with two target sites, and a 65% decrease with one target site; data not shown). The gene knockdown did not depend on the exact antisense modification chemistry as a fully 2′-O-methylated oligonucleotide worked at least as efficiently as the original chemistry which contained unmodified as well as a mix of LNA and 2′O-methyl modified bases (FIG. 9). In the case of the fully 2′-O-methylated cand45 antisense, a slight ˜25% reduction in target reporter gene activity was already observed in the absence of cand45 overexpression. These results were obtained with mouse embryonic fibroblast cells thereby excluding this to be a human-specific phenomenon or a peculiarity of the 293 cell line (FIG. 9). This response, in which an oligonucleotide sense to the target gene induces gene knockdown, was quite distinct from our own experience (data not shown) and that of many others in the literature where antisense oligos against RNAi-related small RNAs such as microRNAs relieve target gene repression. We therefore refer to this phenomenon as ‘sense-induced trans-silencing’, or SITS. The specificity of SITS was confirmed by the use of 3 additional control oligonucleotides (FIG. 6C). Cand45 antisense-mediated gene knockdown was moreover RNAi-related as it could be modulated by Argonaute overexpression (FIG. 6D) similar to how Argonaute overexpressions affect the silencing of perfectly complementary small RNA target genes (Diederichs et al. 2008): Argonaute 2 enhanced the anti-45 knockdown effect (to >90% silencing), while the non-slicing Argonautes 1, 3, and 4 each relieved it (to ˜50-55% silencing), probably by competition with Argonaute 2 for either the target site and/or small RNA.
Argonaute 2 Uniquely Selects for Perfectly Complementary dsRNA of ˜21 Bp

To elucidate the mechanism of SITS, we considered potential changes in Argonaute loading following sense oligo addition. Both the original cand45 tsRNA and a cand45 version extended by 2 nucleotides at its 5′ end were tested in combination with various complementary sense oligonucleotides differing slightly in the double-strand RNA structure that would be reconstituted (FIG. 7). Strikingly, and in agreement with the notion that Ago 2-loaded small RNAs are the main effectors of the trans-silencing of perfect complementary target genes, the addition of all complementary sense oligonucleotides enhanced Ago 2 loading by 2 to 4-fold. By contrast, Ago 3 and 4 loading which was very efficient for the single-stranded cand45 (−50% on input was immunoprecipitated) was not further enhanced by sense oligo addition, and in some cases appeared to be slightly impaired by it. Ago 1 was somewhat intermediate with no, or slightly enhanced loading following sense oligonucleotide addition. Of note, the sense oligonucleotides that enhanced Ago 2 loading in this experiment were fully 2′-O-methylated. As such a modification pattern would be predicted to interfere with passenger strand cleavage by Ago 2 (Leuschner et al. 2006), our results suggest that the relative ability of Ago2 for loading fully duplexed small RNAs is independent of its Slicer activity. These findings are consistent with the relative Ago-association patterns observed for microRNAs and tsRNAs and suggest a rule whereby the degree of complementarity of the small RNA loading substrate determines the efficiency with which it is loaded onto the various Argonautes: extensively duplexed small RNAs into preferentially Ago 2, and somewhat Ago 1, and less stably duplexed and single-stranded RNAs into Agos 3 and 4 (Table 2). While double-strandedness appeared to be the main determinant for Ago 2 loading and SITS efficiency, a more extensive screen of guide strand-sense oligo combinations revealed some differences in SITS efficiencies depending on the exact duplex structure (FIG. 8). Although the nature of 5′ and 3′ overhangs could significantly impact SITS, SITS efficiency only poorly correlated with siRNA design rules such as 2 nucleotide 3′ overhangs. The length of the guide strand, however, had a more obvious impact on SITS efficiency with 20 and 22 nucleotides guides functioning better than 18 and 24 nucleotide guides. Interestingly, Argonaute 2 appeared to discriminate against guide RNAs of unusual length through its PAZ domain as deleting the PAZ domain of Ago 2 conferred onto it the ability to immunoprecipitate cand45-derived small RNAs of various sizes (data not shown). This further correlated with impaired Ago loading, in the case of the original 20 nt cand45 loss of sense-enhanced Ago loading, and ultimately SITS efficiency. To exclude that the heterogeneous small RNAs isolated with the Ago2 PAZ mutant was an experimental artifact as a result of the fact that the PAZ domain binds the 3′ end of the guide RNA (Lingel et al., 2003) and the absence of the PAZ domain might render a bound small RNA susceptible to RNase degradation, we re-hybridized the blot for endogenous microRNAs. This showed that such an artifact was unlikely since only single microRNA bands were observed, while microRNA association was similarly diminished in the PAZ deletion mutant. The Argonaute distributions of microRNAs were largely unaffected in the presence of SITS. Interestingly, however, in a number of instances there was a noticeable decrease in Ago 2 association, potentially the result of competition between the SITS guide RNA and microRNAs for Ago 2 (FIGS. 7A and B, down arrows).

tsRNA Levels Correlate with microRNA and siRNA Silencing Activities

To further test whether tsRNAs could affect the function of other classes of small RNAs, we sought to evaluate microRNA and siRNA silencing activity following the modulation of tsRNA levels. To find conditions under which tsRNA levels may be changed, we took two approaches. One was based on changing the serum concentration in the cell growth media since tRNA transcription rates had been linked cell proliferation (for review, Marshall and White 2008). Alternatively, we overexpressed the RNA polymerase III transcription factor Brf 1 that had been found to specifically upregulate tRNA transcription in mouse cells (Marshall et al. 2008). Transient Brf 1 overexpression in 293 cells led to a 1.5-2.5 fold increase of tsRNAs cand45 and cand520 (data not shown). At the same time, there was a tendency for microRNAs to be down-regulated. Increasing the serum concentration from 1.5% to 10% had a similar effect on relative small RNA levels: tsRNAs cand45 and cand520 were increased at high serum concentrations, while microRNAs were largely unchanged (data not shown), the latter finding being consistent with what had been reported for microRNA levels of subconfluent, serum-starved cells (Hwang et al. 2009). We note that the serum experiments were performed in HCT cells as these proved to be more resistant to outwardly adverse effects of low serum conditions. In both cases, changes in the levels of the type I tsRNAs cand14 and 33 were not consistently observed, although it is possible that some type I tsRNAs not tested for were elevated (data not shown).

We next assessed whether small RNA silencing activity was changed with Brf 1 overexpression or changes in serum concentrations. Depending on the type of competition between tsRNAs and other small RNAs, we would expect the outcome of such experiments to be quite complex. For example, increased tsRNAs would primarily compete with other small RNAs for Ago3 and 4 incorporation. The Argonaute distributions of these small RNAs may therefore be shifted towards Ago 1 and 2, the degree of which, however, would depend on the relative Argonaute affinities and abundances of each particular small RNA. Since tsRNAs will also compete with other small RNAs for Ago 1 and 2, the net effect of such a re-distribution may be muted. In this way, Ago3 and 4 may serve as buffers ensuring e.g. relatively constant microRNA occupancies of Agos 1 and 2 which may be particularly important for their function. It is also possible that tsRNAs interact with the RNAi machinery upstream of Argonautes which in turn may make them more available for small RNAs that enter RNAi at the Argonaute stage, e.g. synthetic siRNAs. As a final example of the potentially complex functional outcomes of modulating small RNA levels, although the absolute abundance of a given small RNA may be increased, if it has a higher affinity for the less efficiently silencing Argonautes 3 and 4 and there was competition for target sites, then overall silencing may also be inhibited (both cleavage and translational silencing pathways).

Brf 1 was overexpressed in 293 cells and this was followed by the introduction of various Renilla luciferase reporter genes that only differed in their small RNA target sites located in the 3′ UTRs. Firefly luciferase on the same plasmid and a Renilla luciferase construct containing mock target sites were used for normalization. There was no or little effect of Brf 1 overexpression on the type I cand14, cand33, and type II cand45 reporters, consistent with no changes and/or preferential Ago 3 and 4 incorporation for these tsRNAs. For the microRNA reporters we chose let-7a and miR-20. For each microRNA, two reporters were constructed: one version with three tandem perfect complementary target sites (‘PM’) for assessing slicing activity, and a corresponding translational reporter version with complementarity for the microRNA seeds, but mismatched downstream thereof, including at positions 10 and 11 which is predicted to abrogate Ago 2 slicing (‘MM’). Unlike the tsRNA reporters, both let-7 reporters were de-repressed by ˜2.5 fold in the presence of increased tRNA transcription factor Brf 1 (data not shown) while let-7a steady-state levels were not significantly changed (data not shown). This result is consistent with tsRNAs modulating let-7 silencing activity. The miR-20 reporters were slightly affected by Brf-1 overexpression with a ˜50% increase for miR-20PM, and none for miR-20mM. As discussed above, such differences between reporters for different microRNAs could be due to various factors such as possible differences in loading efficiencies, absolute abundances and dose sensitivities of the reporter genes. Increased tsRNA levels in the presence of higher serum concentrations were accompanied by ˜2-fold de-repressions of both let-7a and miR-20 perfect match reporters, whereas the corresponding translational reporters that differed from the perfect match reporters by only a few nucleotides were unaffected (data not shown). To exclude that inefficient silencing of the translational microRNA reporters in HCT cells was responsible for lack of de-repression in 10% serum, we co-transfected a let-7a antisense inhibitor and observed a ˜75% upregulation of the reporter gene activity. Interestingly, this simple antisense-mediated inhibition of let-7a underestimated the true extent of let-7a translational repression of the reporter, as the addition of a type of microRNA inhibitor with a region complementary to the microRNA flanked on both sides by small hairpins and that had been reported to be more efficient than simple antisense for microRNA inhibition (Vermeulen et al. 2007) increased reporter gene activity by over 5-fold compared to a control inhibitor directed against miR-20 (data not shown). We conclude that the lack of upregulation of the translational microRNA reporters was not due to their inefficient response to microRNAs. We speculate that given the little changed steady-state level of let-7a at 1.5% versus 10% serum, the difference between the PM and MM reporters is due to a re-distribution of let-7a between the Argonautes (more in Ago2 at 1% than 10% which is predicted to mainly affect PM, and not MM reporters).

To test the effect of Brf-1 overexpression on siRNA efficacy, we transfected three different siRNAs that were directed against the endogenously expressed gene RALY both at a low (500 μM) and a high (50 nM) concentration, and then measured RALY mRNA levels by qRT-PCR (data not shown). ‘No siRNA’ and actin mRNA served for control and normalization purposes, respectively. As expected and in support of the sensitivity of the assay, in each case increased siRNA concentrations more effectively silenced RALY under standard tissue culture conditions of 10% serum. Interestingly, Brfl overexpression in 293 cells increased siRNA efficacy. In the case of RALY siRNA #3, the fold knockdown was increased from about 2- to 4-fold at the low, and from about 2.5 to 5-fold at the high siRNA concentration. It is unlikely that this is due to Brf 1 increasing transfection efficiency, because a) plasmid DNA transfection was not affected by Brfl overexpression based on no significant changes in non-targeted firefly luciferase expression (data not shown), and b) silencing at 500 μM of siRNA#3 with Brf 1 overexpression exceeded its performance at the 100-fold higher concentration of 50 nM in the absence of Brf 1 over-expression. Instead, we speculate that Brf 1 over-expression further shifts incorporation of the perfectly duplexed siRNA into Ago 2 relative to the other Argonautes resulting in reduced competition for the target mRNA. The results are also consistent with tsRNAs competing for components of small RNA silencing upstream of Argonaute loading, thus increasing the availability of Argonautes for the siRNAs. Essentially the same increase in siRNA silencing efficiency was observed in HCT cells when raising serum concentrations from 1% to 10% (data not shown), strengthening the notion that these effects were mediated by tsRNAs and not other changes taking place in the presence of Brfl overexpression or variations in serum concentrations.

### Discussion

Our discovery that there exists in mammals a class of tRNA-derived small RNAs that interacts with the RNAi machinery prompted investigations that contribute to our nascent understanding of the differential properties of the four human Argonautes. Since tsRNAs are not well conserved on a sequence level, yet are observed from yeast to Man (Girard et al. 2006; Babiarz et al. 2008; Buhler et al. 2008), we introduce the concept of how the function of one class of naturally occurring small RNAs may be to regulate the global activity of another class of small RNAs. Global suppression of microRNA abundance and function has been noted for proliferative diseases such as cancer (Lu et al. 2005) and in response to changes in cell densities (Hwang et al. 2009). It will be of interest to test whether these phenomena converge on signaling pathways regulating the expression of tsRNAs. A general, but modest decrease of microRNA abundance as seen in cancer (Lu et al. 2005) may be explained by increased turnover of microRNAs that cannot be loaded when increased levels of tsRNAs are present. Indeed, increased tRNA transcription rates and steady-state levels have been linked to cancer (Marshall and White 2008; Pavon-Eternod et al. 2009). Given the critical roles of microRNAs in cancer (Iorio and Croce 2009), it is tempting to speculate that the outcome of such competition between tsRNAs and microRNAs may at least partly explain the link between tRNA overexpression and cancer. In fact, modest changes in steady-state levels of microRNAs could actually mask still more significant functional differences given that competition would involve four, also biochemically distinct Argonaute proteins in humans. This concept of small RNA class competition, corroborated by our own studies on microRNA and synthetic siRNA efficacies under conditions of varying tsRNA concentrations, is also based on the well documented limited capacity of RNA silencing which is subject to auto-regulatory feedbacks and in which Argonautes compete with each other for small RNAs and targets (Grimm et al. 2006; Diederichs et al. 2008; Forman et al. 2008; Han et al. 2009). Clearly, for this reason alone, it is important to identify and characterize all RNAs that interface with the RNA silencing pathways, particularly many of the small RNAs that had previously been sequenced, but have largely been ignored as degradation products of abundant RNA of no particular functional consequence, even when found to co-immunoprecipitate with Argonaute and Piwi proteins. The inability to detect many of them as distinct 21-22 nucleotide small RNAs in conventional Northern blots and to match a large number of them to the genome, recently found to be largely the result of RNA modifications common to highly abundant RNA (Ebhardt et al. 2009), may have contributed to this neglect. Careful bioinformatic analyses, however, have started to reveal that some of the small RNAs derived from abundant non-coding RNAs, particularly tRNAs, are the product of specific processing by endonucleases such as Dicer (Babiarz et al. 2008; Cole et al. 2009).

Additional evidence for competition between tsRNAs and other classes of small RNAs not only in humans comes from the observation that the loss of DGCR8 and Dicer in mouse embryonic stem cells was accompanied by increased tsRNA levels (Babiarz et al. 2008). In fission yeast, deletion of a component of an RNA turnover complex was associated with an increase in tRNA- and rRNA-derived small RNAs that were bound to Argonaute (Buhler et al. 2008). The fact that only little tsRNA was bound to Argonaute in fission yeast may reflect the existence of only one Argonaute protein in this organism. It therefore lacks other Argonautes that could buffer the major functional Argonaute from unwanted small RNAs. That a similar interaction between exosome RNA turnover and RNAi may also exist in mammals is suggested by the observation that elevated beta-globin gene cluster intergenic transcription following Dicer knockdown was particularly noted in cells treated with trichostatin A, a histone deacetylase inhibitor (Haussecker and Proudfoot 2005). As the global increase in histone acetylation is predicted to globally increase intergenic transcription, this may overwhelm other RNA turnover mechanisms and make these transcripts increasingly accessible for RNAi-related turnover.

The functional consequence of such competition is predicted to be quite complex, particularly in organisms with multiple Argonautes each with slightly different expression patterns and relative specificities for the different classes of small RNAs and even for different small RNAs within a class. Moreover, as shown here, competition between classes of small RNAs may also be a dynamic property depending on the physiologic state of the cell. tsRNAs differ in a number of respects from microRNAs and can be grouped into two sub-classes based on differences in biogenesis and biological activity (Table 1). We demonstrate here that the Microprocessor-independent, Dicer-dependent type I tsRNAs, when appropriately pre-treated, can in fact be observed as distinct 5′-phosphorylated, 3′-hydroxylated small RNAs that are incorporated into Argonautes and have trans-silencing capacity. Unlike microRNAs, however, neither tsRNA sub-class associated with Mov10 and tsRNAs were essentially restricted to the cytoplasm. Type II tsRNAs, as exemplified by cand45, are generated by RNaseZ cleavage at the discriminator base of tRNAs to generate the phosphorylated 5′ end and by RNA polymerase III termination leaving a stretch of uracils at the 3′ end. As these processes are thought to occur in the nucleus and quality control mechanisms exist to ensure that only properly processed, mature tRNAs are exported (Lund and Dahlberg 1998), this suggests type II tsRNAs to be efficiently exported following synthesis. Since some Argonautes, like microRNAs (Hwang et al. 2007), have been demonstrated in the nuclei of mammalian cells (e.g. Rudel et al. 2008) and are able to shuttle between the nucleus and cytoplasm (Guang et al. 2008; Weinmann et al. 2009), Argonautes themselves may be responsible for the export and cytoplasmic localization of type II tsRNAs, possibly through their association with the tsRNAs soon after RNA polymerase III transcription termination. Alternatively, type II precursor tRNAs may escape nuclear quality control and are processed by the less well defined pool of cytoplasmic RNaseZ (Elbarbary et al. 2009).

Overlap between various non-coding RNA biogenesis pathways is not uncommon. In this regard, the type II tsRNAs are reminiscent of the RNaseZ-mediated separation of dicistronic RNAs into upstream tRNAs and downstream snoRNAs in plants (Kruszka et al. 2003). RNaseZ has also been recently reported to act on the nascent long non-coding human MALAT1 precursor RNA (Wilusz et al. 2008), thereby generating the mature 3′ end of MALAT1 RNA and liberating a downstream cytoplasmic tRNA-like small RNA. The only relatively recently discovered RNaseZ may therefore function in a much wider array of biological pathways than previously anticipated. A link between snoRNA and microRNA biogenesis was established by Ender and colleagues who described a human gene originally thought to function only as a snoRNA, but that was then found to be also processed in a Microprocessor-independent, Dicer-dependent manner into an Argonaute-associated silencing small RNA (Ender et al. 2008). Overall, the evolution of small RNA biology appears to be highly experimental and flexible in that various mechanisms that can generate hairpins and/or 5′-phosphorylated small RNAs may all enter into RNAi-related pathways.

Although it remains to be elucidated why exactly Argonaute 2 is genetically the most important Argonaute for mammalian cell viability, its importance may also be reflected in the relatively high molecular selectivity of guide RNA loading (FIG. 7C). Both the Argonaute distribution of microRNAs and tsRNA and the mechanism of action of the sense-induced trans-silencing phenomenon show that Argonaute 2 has a preference for 20-22 base-pair, fully duplexed dsRNAs relative to the other Argonautes. Deletion studies further indicated that the PAZ domain contributes to this size and structural selectivity. By contrast, particularly Argonautes 3 and 4 which lack slicing capacity and have intrinsically less translational silencing capacity (Su et al. 2009) may act as buffers soaking up unstructured, especially small single-stranded RNAs. They may thus serve to protect the cells from adventitious degradation products, therefore preventing them from having widespread impact on cellular gene expression through guiding off-targeting. This property of Argonautes 3 and 4 is also of interest for the application of single-stranded RNAs to induce RNAi (ssRNAi), since as the cand45 example shows, the mere presence of a small 5′-phosphorylated RNA is not sufficient for effective trans-silencing. Because RNAi can be elicited by the transfection of single-stranded RNAs (Martinez et al. 2002), small RNA biogenesis may also play a role in determining the small RNA Argonaute distribution pattern. The germline-restricted (primary) piRNAs are another example of a small RNA population that is apparently generated from single-stranded precursor RNAs with no obvious secondary structures via an unknown mechanism that, however, also does not seem to involve Dicer (Vagin et al. 2006), yet loads into Argonaute family proteins. While piRNAs in lower eukaryotes serve to control transposon activity by a so-called ping-pong mechanism, their function and molecular mechanism of action is less well understood in mammals (for review: Aravin et al. 2007). Of note, ˜6% of piRNA complexes isolated from mouse testes contained tRNA-derived small RNAs, yet were essentially entirely depleted of ribosomal and micro-RNAs (Girard et al. 2006).

Given that RNAi efficiency is determined by siRNA-guided slicing of target mRNA by Argonaute 2 (Liu et al. 2004), our results further highlight the importance of achieving comparatively efficient Argonaute 2 loading. Differential duplex end stabilities and consequently biased passenger-guide strand loading is thought to be the single most important determinant for siRNA efficacy (Khvorova et al. 2003; Schwarz et al. 2003). This is surprising, however, since absolute Argonaute 2 occupancy of a passenger strand derived from a highly abundant siRNA should still be higher than that of the preferred guide strand from a much less abundant siRNA, yet silencing efficiencies are not correlated with absolute Argonaute 2 occupancy. We therefore speculate that the improved performance of asymmetric siRNAs is due to their relative efficient loading into Agog rather than their absolute ability to be loaded into Argonautes in general. It is then the ability of Ago2 to rapidly cleave the passenger strand (Matranga et al. 2005; Rand et al. 2005) of asymmetric siRNAs that is ultimately responsible for the observed strand bias. Passenger strands of siRNAs that are equally well recognized by all Argonautes, including non-slicing Agos 1, 3, and 4 should be more stable and such siRNAs should therefore exhibit less apparent strand bias. Poor siRNA performance may thus result from competition for the target with the non-slicing Argonautes. This suggests relative Ago2 loading efficiency to be an important consideration for siRNA design.

An unanticipated finding from our studies were that despite the apparent lack of classical trans-silencing activity by cand45, there was robust, >80% down-regulation of a cand45 reporter gene in cells over-expressing cand45 upon the addition of an oligonucleotide antisense to cand45, i.e. sense to the target gene. This is a quite unusual response to oligonucleotides that are antisense to small guide RNAs as this usually relieves, but does not induce gene repression. We show that reconstitution of a fully duplexed siRNA that is now preferentially loaded into Ago 2 is responsible for this phenomenon. In addition for exploiting this system to learn about Argonaute substrate specificities, equally exciting is the prospect of harnessing this mechanism as a new type of RNA silencing tool in which genes can be silenced by the addition of an oligonucleotide with sense polarity to the target gene. This could not only be useful for studying gene function in vitro, but it may be particularly valuable for knocking down genes in vivo for target validation and therapeutic purposes by combining the relative ease and simplicity of delivering unformulated single-stranded oligonucleotides to organs like the liver and spleen to, in a temporally regulated manner, tap into the inherently more potent RNAi pathway once inside cells. The ability to use at least two different modification chemistries raises hopes that the pharmacological requirements for in vivo applications will not be limiting.

### Example 3

### Cand45 Vector

### Sequence and Construction

In a first step, we cloned out the cand45 gene with primers annealing about 370 bp upstream of the mature cand45 tRNA and at the downstream Pol III terminator site:

Upstream primer:
cttaaAAGCTTaagcttCTCTCGCAGAAATGCCAAAT
Downstream primer:
cttaatctagaAAAAAAAtgGTCTTCAGTGAAGCGAAGACgcagggTTC
GAACCTGCGCGGGGAGAC

Contained in the upstream and downstream primers were Hind III and XbaI restriction sites for cloning into a standard cloning vector (pCR11 in this case). Also contained within the downstream primer are two recognition sites for the type II restriction enzyme BbsI that are facing outward so that Bbs 1 treatment will drop out a fragment that can be replaced with the noncoding RNA of choice, e.g. a small RNA. The fragment that is dropped out in this exemplary cloning vector includes some sequences upstream of the RNaseZ site, i.e. from the 3′ end of the mature cand45 tRNA. In our experiments, we generally put these sequence elements back in, but such a cloning vector would also allow modifying all the sequences downstream of the box B, including the RNaseZ site. This is, however, just one of many cloning strategies known in the art that can be used for a cand45 expression system, some of which do not involve restriction enzymes at all.

Partial sequence of the cand45 vector is shown in FIG. 10.

### REFERENCES

Aoki, K. et al. (2007). EMBO J. 26: 5007-5019.
Aravin, A. A., et al. (2007). Science 318: 761-764.
Azuma-Mukai, A., et al. (2008). Proc Natl Acad Sci USA 105: 7964-7969.
Babiarz, J. E., et al. (2008). Genes Dev 22: 2773-2785.
Berezikov, et al. (2007). Mol Cell 26: 328-336.
Buhler, M., et al. (2008). Nat Struct Mol Biol 15: 1015-1023.
Cao, D., et al. (2009). RNA 15: 1971-1979.
Cole, C., et al., (2009). RNA 15: 2147-2160.
Cummins, J. M., et al., Proc Natl Acad Sci USA 103: 3687-3692.
Diederichs, S., Haber, D. A. (2007). Cell 131: 1097-1108.
Diederichs, S., et al. (2008). Proc Natl Acad Sci USA 105: 9284-9289.
Dubrovsky, E. B., et al. (2004). Nucleic Acids Res 9: 255-262.
Ebhardt, H. A., et al. (2009). Nucleic Acids Res 37: 2461-2470.
Ender, C., et al. (2008). Mol Cell 32: 519-528.
Farazi, T. A., et al. (2008). Development 135: 1201-1214.
Forman, J. J., et al. (2008). Proc Natl Acad Sci USA 105: 14879-84.
Ghildiyal, M., Zamore, P. D. (2009). Nat Rev Genet. 10: 84-108.
Girard, A., et al. (2006). Nature 442: 199-202.
Grimm, D., et al. (2006). Nature 441: 537-41.
Guang, S., et al. (2008). Science 321: 537-541.
Han, J., et al. (2009). Cell 136: 75-84.
Haussecker, D., Proudfoot, N. J. (2005). Mol Cell Biol 25: 9724-9733.
Haussecker, D., et al. (2008). Nat Struct Mol Biol 15: 714-721.
Hock, J., et al. (2007). EMBO Rep 8: 1052-1060.
Hock, J., Meister, G. (2008). Genome Biol 9: 210.1-210.8
Hwang, H. W., et al. (2007). Science 315: 97-100.
Hwang, H. W., et al. (2009). Proc Natl Acad Sci USA 106: 7016-7021.
Iorio, M. V., Croce, C. M. (2009). J Clin Oncol 27: 5848-5856.
Janowski, B. A., et al. (2006). Nat Struct Mol Biol 13: 787-792.
Kawaji, H., et al. (2008). BMC Genomics 9: 157.
Khvorova, A., et al. (2003). Cell 115: 209-216.
Kim, D. H., et al. (2006). Nat Struct Mol Biol 13: 793-797.
Kim, V. N., et al. (2009). Nat Rev Mol Cell Biol 10: 126-139.
Kirino, Y., Mourelatos, Z. (2007). Nat Struct Mol Biol 14: 347-348.
Kok, K. H., et al. (2007). J Biol Chem 282: 17649-17657.
Kruszka, K., et al. (2003). EMBO J. 22: 621-632.
Leuschner, P. J., et al. (2006). EMBO Rep 7: 314-320.
Lingel, A., et al. (2003). Nature 426: 465-469.
Liu, J., et al. (2004). Science 305: 1437-1441.
Lund, E., Dahlberg, J. E. (1998). Science 282: 2082-2085.
Marshall, L., et al. (2008). Cell 133: 78-89.
Marshall, L., White, R. J. (2008). Nature Rev Cancer 8: 911-914.
Martinez, J., et al. (2002). Cell 110: 563-574.
Matranga, C., et al. (2005). Cell 123: 607-620.
Mayer, M., et al. (2000). Biochemistry 39: 2096-2105.
Meister, G., et al. (2004). Mol Cell 23: 185-197.
Meister, G., et al. (2005). Curr Biol 15: 1-7.
Mi, S., et al. (2008). Cell 133: 116-127.
Nashimoto, M., et al. (1999). Nucleic Acids Res 27: 2770-2776.
O'Carroll, D., et al. (2007). Genes Dev 21: 1999-2004.
Pak, J., Fire, A. (2007). Science 315: 241-244.
Pavon-Etemod, M., et al. (2009). Nucleic Acids Res 37: 7268-7280.
Rand, T. A., et al. (2005). Cell 123: 621-629.
Rudel, S., et al. (2008). RNA 14: 1244-1253.
Schwarz, D. S., et al. (2003). Cell 115: 199-208.
Seto, A. G., et al. (2007). Mol Cell 26: 603-609.
Su, H., et al. (2009). Genes Dev 23: 304-317.
Tam, O. H., et al. (2008). Nature 453: 534-538.
Tomari, Y., et al. (2007). Cell 130: 299-308.
Vagin, V. V., et al. (2006). Science 313: 320-324.
Vermeulen, A., et al. (2007). RNA 13: 723-730.
Wang, Y., et al. (2008). Nature 456: 921-926.
Watanabe, T., et al. (2008). Nature 453: 539-543.
Weinmann, L., et al. (2009). Cell 136: 1-12.
Wilusz, J. E., et al. (2008). Cell 135: 919-932.
Yu, B., et al. (2005). Science 307: 932-935.
Zamore, P. D., Haley, B. (2005). Science 309: 1519-1524.
Zhang, X., et al. (2009). Biochim Biophys Acta 1789: 153-159.

We claim:

1. A method to inhibit the expression of a target gene in a cell, the method comprising:
(a) introducing a first single stranded RNA into a cell, said first single stranded RNA comprising a ribonucleotide sequence which is antisense to and complementary to a nucleotide sequence of a target gene, wherein said first single stranded RNA is transcribed from a DNA comprising a RNA polymerase III initiation site;
(b) separately introducing into said cell a second single stranded RNA comprising a ribonucleotide sequence complementary to a portion of the ribonucleotide sequence of said first single stranded RNA; and
(c) forming a double stranded RNA comprising said first single stranded RNA and said second single stranded RNA, said double-stranded RNA inhibiting the expression of the target gene.

2. The method of claim 1, wherein said first single stranded RNA comprising a 5′-phosphate.

3. The method of claim 1, wherein said first single stranded RNA comprising at least 2 continuous uracils at the 3′-end

4. The method of claim 1, wherein said DNA comprises an RNase recognition site.

5. The method of claim 4, wherein said RNase is RNase Z.

6. The method of claim 1, wherein said DNA comprises an RNase polymerase III terminator.

7. The method of claim 1, wherein said first single stranded RNA is transcribed from a DNA by an RNA polymerase III.

8. The method of claim 1, wherein the expression of said first single stranded RNA is driven by a cand45 promoter or a derivative thereof.

9. The method of claim 2, wherein the 5′-phosphate of said first single stranded RNA is generated by the cleavage of an RNase.

10. The method of claim 1, wherein said first single stranded RNA has a length from 18 to 25 nucleotides.

11. The method of claim 3, wherein said first single stranded RNA comprises 2 to 6 continuous uracils at the 3′ end.

12. The method of claim 1, wherein said first single stranded RNA does not possess substantial gene silencing activity without forming said double-stranded RNA with said second single stranded RNA.

13. The method of claim 1, wherein said double-stranded RNA inhibits expression of the target gene through the RNA interference pathway.

14. The method of claim 1, wherein said second single stranded RNA has a length of 14 to 25 nucleotides.

15. An expression vector comprising:
(a) a first DNA sequence comprising an RNA polymerase III initiation site;
(b) a second DNA sequence located downstream of said first DNA sequence, said second DNA sequence comprising an RNase recognition site; and
(c) a third DNA sequence located downstream of said second DNA sequence, said third DNA comprising at least three continuous thymines followed by an RNA polymerase III terminator.

16. The expression vector of claim 15, further comprising a fourth DNA sequence corresponding to the nucleotide sequence of a target gene, said fourth DNA sequence located between said second DNA sequence and said third DNA sequence.

17. The expression vector of claim 15, wherein said RNase is RNase Z.

18. A host cell derived from a cell transfected with the expression vector of claim 15.

19. An animal comprising the host cell of claim 18.

20. A method of generating a single stranded RNA against a target gene, the method comprising:
(1) transcribing a parent RNA from an expression vector with an RNA polymerase III, said parent RNA comprising at least 2 continuous uracil at the 3′-end; and
(2) cleaving said parent RNA with an RNase Z to generate a single stranded RNA having a length of 18 to 25 nucleotides, said single stranded RNA comprising a 5′ phosphate and is antisense to and complementary to a portion of the nucleotide sequence of a target gene, wherein said target gene does not encode a tRNA.

21. An expression vector comprising a cand45 promoter or a promoter derived thereof.

22. The expression vector of claim 21, further comprising:
(a) a first DNA sequence comprising an RNA polymerase III initiation site; and
(b) a second DNA sequence located downstream of said first DNA sequence, said second DNA sequence comprising an RNase recognition site.

23. The expression vector of claim 21, wherein said cand45 promoter comprises 1-373 of the nucleotide sequence depicted in FIG. 10B, a fragment of the 1-373 of the nucleotide sequence depicted in FIG. 10B, or a sequence that is 60%, 70%, 80%, 90%, 95% or more identical to 1-373 of the nucleotide sequence depicted in FIG. 10B.
